(19) United States
(12) Patent Application Publication
(10) Pub. No.: US 2025/0210207 A1
(43) Pub. Date: Jun. 26, 2025

(54) Retrieval-Augmented Synthetic Test Claim Generation

(71) Applicant: Magnum Transaction Sub, LLC, Newtown Square, PA (US)

(72) Inventors: Akshay Sharma, Santa Cruz, CA (US); Kartik Thakore, San Francisco, CA (US)

(73) Assignee: Magnum Transaction Sub, LLC, Newtown Square, PA (US)

(21) Appl. No.: 18/990,262

(22) Filed: Dec. 20, 2024

Related U.S. Application Data
(60) Provisional application No. 63/613,593, filed on Dec. 21, 2023.

(51) Int. Cl. G16H 70/20; G16H 40/20

(52) U.S. Cl. CPC G16H 70/20; G16H 40/20

(57) ABSTRACT

Techniques for generating synthetic test claims for medical procedures are provided. In one example, a transformer model executed on a computer system, and trained on healthcare contexts, receives a medical technical document comprising text data that defines a context that is required for approval of a medical procedure and generates one or more embeddings for the medical technical document. Using the one or more embeddings, the computer system retrieves one or more related medical technical documents and one or more sample claims and inputs them with the medical technical document to a claim generation machine learning model to generate one or more synthetic claims that each comprise a reimbursement request for the medical procedure and are usable to test an accuracy of program code that analyzes claims for compliance with the medical technical document.

### CROSS-REFERENCE TO RELATED APPLICATIONS

This application claims priority to U.S. Provisional Patent Application No. 63/613,593, filed on Dec. 21, 2023, entitled “Pseudo-Code Generation via Application Plug-In,” the disclosure of which is hereby incorporated by reference in its entirety for all purposes.

### BACKGROUND

Compliance with rules set forth in the medical field is conventionally a manual process. Such compliance is exceptionally difficult and time consuming. Regulations set forth by a governing entity, such as Medicare, is reviewed by human analysts to draft technical documents of how such rules and regulations should be implemented. This technical specification can then be used by an entity to determine how data, such as medical claims, should be processed.

### SUMMARY

In some embodiments, a method of generating synthetic claims for medical procedures is provided. The method may include receiving a medical technical document comprising text data that defines a context that is required for approval of a medical procedure at a transformer model executed on a computer system. The transformer model may be trained on healthcare contexts. The method may further include generating, by the transformer model executed on the computer system, one or more embeddings of the medical technical document. The method may further include retrieving, by the computer system, using the one or more embeddings, one or more related medical technical documents and one or more sample claims. The one or more sample claims may be related to the related medical technical documents. The method may further include inputting, by the computer system, to a claim generation machine learning model, the medical technical document, the one or more related medical technical documents, and the one or more sample claims to generate one or more synthetic claims that each comprise a reimbursement request for the medical procedure. The method may further include receiving, by the computer system from the claim generation machine learning model, the one or more synthetic claims. The one or more synthetic claims may be usable to test an accuracy of program code that analyzes claims for compliance with the medical technical document.

In some embodiments, the one or more embeddings generated by the transformer model are dense vector representations in a continuous vector space. In some embodiments, retrieving the one or more related medical technical documents comprises performing a similarity search within an embedding datastore of medical technical documents using the one or more embeddings of the medical technical document. In some embodiments, retrieving the one or more related medical technical documents comprises: determining a similarity score for each of a plurality of medical technical documents, wherein the similarity score for a respective medical technical document of the plurality of medical technical documents is based on a comparison between the one or more embeddings of the medical technical document and one or more embeddings of the respective medical technical document; and selecting the one or more related medical technical documents from the plurality of medical technical documents based on a threshold similarity score.

In some embodiments, the transformer model is trained using contrastive learning on pairings between similar and dissimilar medical procedures and diagnoses identified from historical medical technical documents. In some embodiments, the context defines one or more criteria that are required for approval of the medical procedure. In some embodiments, the one or more criteria comprise a first medical diagnosis that justifies the medical procedure. In some embodiments, the one or more synthetic claims comprise a first claim that justifies the medical procedure with the first medical diagnosis.

The method may further include retrieving, using the one or more embeddings, a procedure code for the medical procedure and a diagnostic code for the medical diagnosis and providing the procedure code and the diagnostic code to the claim generation machine learning model. In some embodiments, the one or more synthetic claims comprise a first claim that justifies the medical procedure with a second medical diagnosis that is not included in the one or more criteria. In some embodiments, the one or more synthetic claims comprise a first set of synthetic claims that satisfy the context and a second set of synthetic claims that do not satisfy the context. In some embodiments, each of the one or more synthetic claims is generated with a label indicating whether it satisfies the context or not.

The method may further include executing the program code on the one or more synthetic claims to produce determinations, for each respective synthetic claim of the one or more synthetic claims, indicative of whether the respective synthetic claim complies with the medical technical document. The method may further include determining, based on a comparison between the results generated by the program code and the label generated with each of the one or more synthetic claims, the accuracy of the program code.

In some embodiments, a retrieval augmented synthetic test claim generation system is provided. The retrieval augmented synthetic test claim generation system may include one or more processors and a computer-readable storage media storing computer-executable instructions that, when executed by the one or more processors, cause the retrieval augmented synthetic test claim generation system to receive a medical technical document comprising text data that defines a context that is required for approval of a medical procedure at a transformer model. In some embodiments, the transformer model is trained on healthcare contexts. The instructions may further cause the retrieval augmented synthetic test claim generation system to generate, by the transformer model, one or more embeddings of the medical technical document. The instructions may further cause the retrieval augmented synthetic test claim generation system to retrieve, using the one or more embeddings, one or more related medical technical documents and one or more sample claims, wherein the one or more sample claims are related to the related medical technical documents. The instructions may further cause the retrieval augmented synthetic test claim generation system to input, to a claim generation machine learning model, the medical technical document, the one or more related medical technical documents, and the one or more sample claims to generate one or more synthetic claims that each comprise a reimbursement request for the medical procedure. The instructions may further cause the retrieval augmented synthetic test claim generation system to receive, from the claim generation machine learning model, the one or more synthetic claims, wherein the one or more synthetic claims are usable to test an accuracy of program code that analyzes claims for compliance with the medical technical document.

In some embodiments, one or more non-transitory computer-readable storage media are provided. The one or more non-transitory computer-readable storage media may store one or more instructions which, when executed by one or more processors of a retrieval augmented synthetic test claim generation system, cause the one or more processors to receive a medical technical document comprising text data that defines a context that is required for approval of a medical procedure at a transformer model. In some embodiments, the transformer model is trained on healthcare contexts. The one or more processors may be further caused to generate, by the transformer model, one or more embeddings of the medical technical document. The one or more processors may be further caused to retrieve, using the one or more embeddings, one or more related medical technical documents and one or more sample claims, wherein the one or more sample claims are related to the related medical technical documents. The one or more processors may be further caused to input, to a claim generation machine learning model, the medical technical document, the one or more related medical technical documents, and the one or more sample claims to generate one or more synthetic claims that each comprise a reimbursement request for the medical procedure. The one or more processors may be further caused to receive, from the claim generation machine learning model, the one or more synthetic claims, wherein the one or more synthetic claims are usable to test an accuracy of program code that analyzes claims for compliance with the medical technical document.

### BRIEF DESCRIPTION OF THE FIGURES

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates a block diagram of an embodiment of a quality assurance system for testing implementation of rules defined by medical technical documents.

FIG. 2 illustrates a block diagram of an embodiment of a system that uses artificial intelligence accessible via an application plug-in to generate pseudocode and test scenarios for quality assurance testing.

FIG. 3 illustrates a block diagram of an embodiment of a system that uses retrieval augmented artificial intelligence accessible to generate pseudocode and test data for quality assurance testing.

FIG. 4 illustrates a flow diagram for training a transformer to generate embeddings for source documents that capture the relationships between source documents.

FIG. 5 illustrates a flow diagram for generating embeddings of source documents for retrieval augmented pseudocode and test data generation.

FIG. 6 illustrates a sequence diagram that depicts the interactions between components in an embodiment of a retrieval augmented pseudocode and test data generation system.

FIG. 7 illustrates an embodiment of a plug-in implemented in a word processing program to generate pseudocode for quality assurance testing.

FIG. 8 illustrates an embodiment of a plug-in implemented in a word processing program to solicit for feedback based on generated pseudocode for quality assurance testing.

FIG. 9 illustrates an embodiment of a method for generating pseudocode for quality assurance testing.

FIG. 10 illustrates an embodiment of a method for implementing code created based on generated pseudocode for quality assurance testing.

FIG. 11 illustrates an embodiment of a method of generating synthetic claims for a medical procedure based on a medical technical document.

FIG. 12 illustrates an embodiment of a method of generating a medical technical document based on a medical regulation.

FIG. 13 illustrates a block diagram of an embodiment of a computer system according to some embodiments.

### DETAILED DESCRIPTION

As detailed herein, an artificial intelligence (AI) based system, such as using one or more machine learning (ML) models, can be accessed via an application plug-in or dedicated application to improve the speed and accuracy of quality assurance (QA) of programming code-based implementations of technical rule-based documents, such as medical rule implementation specifications or medical technical documents. The use of a plug-in or dedicated application can allow for the technical specification to be accessed and analyzed to create various QA pseudo-code steps that are to be implemented as processor-readable test code along with an indication of the results expected to be generated by such test code. This test code can then be used to perform QA of a code-based rule implementation of the technical document to ensure accuracy of the implementation prior to the code-based rule implementation being used. As part of the process, code testing scenarios with synthetic claims can be created to test the code-based rule implementation using a testing artifact database. Once created and tested, code-based rule implementations can be distributed to one or more clients, which can then use the code-based rule implementations to comply with relevant rules and regulations.

Embodiments described herein address numerous technical challenges associated with using AI based systems to generate medical technical documents, and code-based implementations thereof, that are compliant with complex medical regulations and guidelines, as well as test scenarios that can be used to verify such compliance. One such technical challenge arises due to the closed universe of knowledge upon which generative AI models are trained. Unless such models are constantly retrained as new information becomes available, which would be time and cost prohibitive due to the frequency with which healthcare services and regulations evolve, the information they are able to draw from may be outdated, which can lead to can lead to inaccurate or non-compliant outputs. This can result in the generation of medical technical documents and synthetic claims that do not reflect the latest regulatory requirements and guidelines, potentially causing significant compliance issues and operational inefficiencies.

To address this challenge, embodiments described herein incorporate techniques such as context-aware embeddings and continuous retrieval of relevant information. By generating embeddings that accurately capture the semantic and contextual relationships within existing medical technical documents and regulations, and by leveraging these embeddings to retrieve the most up-to-date related documents and sample claims, the system can ensure that the generated outputs are both current and compliant with the latest regulations. This approach minimizes the need for constant retraining of the generative models, thereby reducing time and cost constraints while maintaining high accuracy and compliance.

Additionally, the use of contrastive learning to train transformer models enhances their ability to differentiate between similar and dissimilar pairs of documents and claims, further improving the quality of the generated embeddings and the resulting synthetic claims. By continuously integrating new information through context-aware retrieval and embedding generation, the system remains adaptable to the evolving landscape of healthcare regulations and guidelines, ensuring that the outputs are always aligned with the most recent standards and best practices.

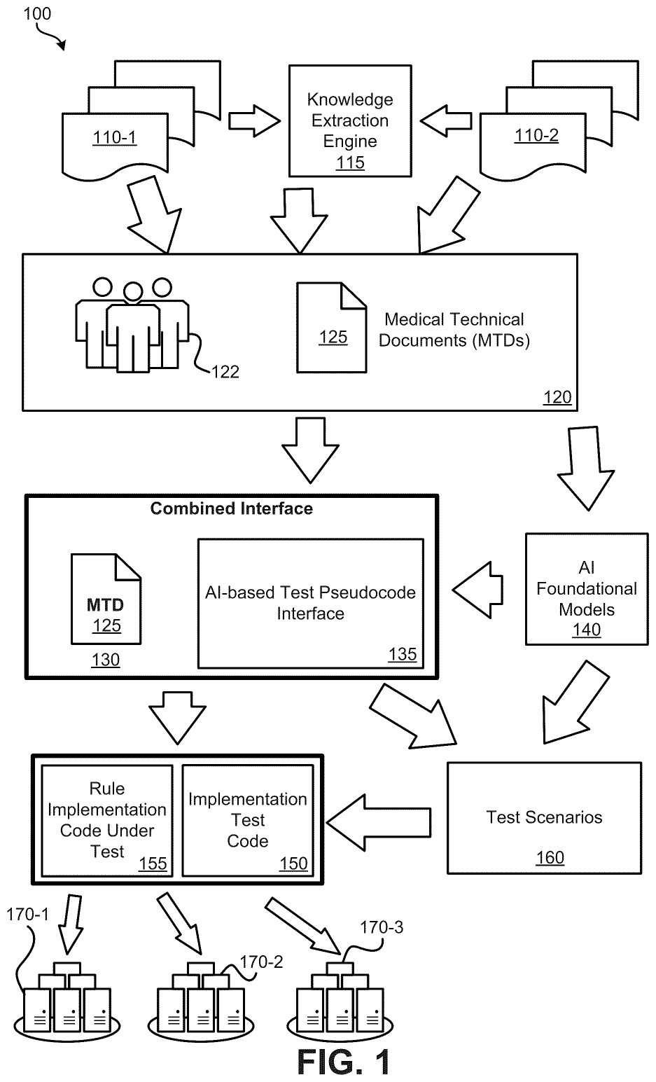

Additional detail regarding these embodiments and additional embodiments are provided in relation to the figures. FIG. 1 illustrates a block diagram of an embodiment of a quality assurance system 100 for generating and testing implementations of rules defined by medical technical documents. System 100 can include one or more computer systems through which medical regulations 110 and medical technical documents (MTDs) are processed. Such computer systems can represent computing hardware executing specialized software or special-purpose processors configured to perform the functions as detailed herein.

Medical regulations 110 (e.g., 110-1, 110-2) refers to regulations and rules set forth by various entities, such as drug companies, government entities (e.g., the Food and Drug Administration, FDA; the Centers for Medicare and Medicaid Services, CMS; state and local health agencies; etc.). These regulations and rules can define the circumstances under which particular medical procedures, treatments, therapies, and drugs should and should not be applied, prescribed, or administered. As a simple example, the recommendation for a particular therapy being used to treat a particular condition may change to be only approved for patients over a defined age and/or for patients who have received a particular diagnosis or combination of diagnoses. These regulations and rules may further define the approved billing procedures and amounts for approved procedures, treatments, and/or therapies.

Updates to medical regulations 110 can be published by their source entities at periodic, semi-periodic, and/or random intervals. For example, CMS transmittals may be published to the CMS website on a monthly basis and/or as they are finalized. To ensure that system 100 stays up-to-date as updates are published, knowledge extraction engine 115 may periodically scan known websites and/or webpages on which updates are published from their sources to identify new and/or updated medical regulations 110. For example, and as described further herein, knowledge extraction engine 115 may periodically visit a list of known website and/or webpages. From an initial webpage, knowledge extraction engine 115 may perform a recursive search for digital documents meeting one or more criteria associated with medical regulations, including searching through pages linked from the initial webpage. Once identified, knowledge extraction engine 115, and/or AI foundational models 140, may determine whether each candidate document represents a medical regulation, or other relevant document.

Medical regulations 110 can be analyzed by one or more medical experts 122 to create one or more MTDs 125. MTDs 125 can define the specific steps and actions to be taken in order to comply with medical regulations 110 and process a claim in accordance with the regulations. Returning to the previous age-based example, while the regulation may simply define a procedure that should not be administered if the patient is below a given age, one of MTDs 125, may be targeted to a process flow for analyzing a claim. Steps could, for example, include: determining a claim type; verify that the procedure is available for the claim type; calculate the patient's age based on the patient's date of birth; deny coverage if age criteria for procedure is not satisfied.

Additionally, or alternatively, knowledge extraction engine 115 and/or AI foundational models 114 may process medical regulations 110 to create MTDs 125. For example, and as described further herein, AI foundational models 140 may include one or more machine learning models trained to generate new MTDs, and/or update existing MTDs, from new and/or updated medical regulations. Given a new or updated medical regulation, knowledge extraction engine 115 may identify similar historical medical regulations and/or related historical MTDs and provide them to AI foundational models 140 along with the new or updated medical regulation. Once generated, the new and/or updated MTDs may be provided to medical experts 122 with the new or updated medical regulation for approval and/or editing.

Claims for medical coverage, referred to herein simply as claims or a claim, may refer to claims submitted by a medical provider for reimbursement of costs associated with one or more procedures and/or services provided by the medical provider to a patient. Claims may be submitted in one or more structured formats with predefined fields for identifying information about a patient, the procedures and/or services provided to the patient, the diagnoses that justify the provisioning of the procedures, or the like. These structured formats typically include fields for standardized coding systems such as International Classification of Diseases (ICD) codes for diagnoses, Current Procedural Terminology (CPT) codes for procedures, and Healthcare Common Procedure Coding System (HCPCS) codes for additional services and supplies. For example, a claim may include multiple line items, each corresponding to a separate procedure and/or service for which the medical provider is seeking reimbursement and the amount for which reimbursement is being sought. Additionally, each line item may include a diagnosis provided as justification for the procedure and/or service provided to the patient.

Upon receipt of a claim, a payer, which could be an insurance company, a government health program, or another entity responsible for the reimbursement, will process the claim to determine the eligibility and appropriateness of the submitted charges. This process may involve the validation of the claim's data with the regulations and rules set forth in medical regulations 110 by the various entities described above. Additionally, or alternatively, validating the claim's data may include cross-referencing with the patient's coverage details, and the application of any relevant policy limits, exclusions, or pre-existing condition considerations.

Once created, MTDs 125 define how a claim for medical coverage should be analyzed for approval, denial, and/or modification in view of the medical regulations. However, in order to analyze claims in an automated fashion, MTDs 125 need to be converted to computer-readable code. This creation of code can be done in either an automated or manual fashion. As part of MTD creation 120, code may be created by medical experts 122, AI foundational models 140, and/or another party that implements MTDs 125. Referring back to the previous example, this code can allow for the claim type to be determined; the procedure to be verified as available for the claim type; calculation of the patient's age; and denial of coverage if the age criteria for procedure is not satisfied.

As described further herein, MTDs 125 may be implemented as computer-readable code that is configured to receive one or more claims as input and generate an output indicating whether the claim as a whole should be paid in full, denied in full, and/or whether individual line items should be paid in full or denied. MTDs may be implemented in one or more programming languages, such as C++, XML, or the like. The computer-readable code may include one or more algorithms that apply the one or more conditions defined in the MTD to the claims. The computer-readable code may further include one or more processes configured to receive claims in one or more different formats and convert the claim into the object model and/or schema accepted by the code for MTDs.

Implementing MTDs 125 and claims as computer-readable code may enable a high volume of claims to be analyzed and processed. However, once created, extensive testing needs to be performed in order to ensure that the code will correctly process either all or the vast majority of claims. False positives or false negatives, (in this case, approving a claim when it should have been denied or denying a claim when it should have been approved) can result in a significant amount of patient and administrative time being expended to correct the error.

As detailed in relation to FIG. 1, combined interface 130 can allow for human readable versions of MTDs 125 to be viewed while AI-based test pseudocode interface 135 (“interface 135”) is being concurrently presented. In some embodiments, interface 135 can provide MTDs 125 to AI foundational models 140 to generate test scenarios that are used to test whether the code created based on MTDs 125 is functioning correctly, especially for edge cases. A simple example of such an edge case would be someone who is the exact correct age, in number of days, to meet the threshold criteria to qualify for a medical procedure under an MTD. Ten, hundreds, or even thousands of test scenarios may be created via interface 135 using AI foundational models 140.

In other embodiments, rather than directly creating test scenarios, interface 135 can be used to create pseudocode that allows a person, such as a QA supervisor, and/or AI foundational models 140 to convert the pseudocode into actual code. By creating pseudocode, the process may be significantly accelerated and made more accurate than relying exclusively on a QA supervisor to create the code without a guide.

The test scenarios created for MTDs 125, directly by AI foundational models 140 from an MTD or based on pseudocode, are output as test scenarios 160. Each test scenario created for an MTD may include one or more claims, each including one or more line items, as described above. Test scenarios generated for a particular MTD may include a subset of positive test scenarios, each including one or more claims and/or one or more line items that satisfy the rules and conditions defined in the MTD, as well as a subset of negative test scenarios, each including one or more claims and/or one or more line items that do not satisfy the rules and conditions defined in the MTD. Each test scenario may be generated with a label or other annotation indicating the result that should be generated by the code.

As described further herein, the claims and associated claim data in a test scenario for a particular MTD may be synthetically generated based on the particular MTD. For example, given an MTD that stipulates reimbursement criteria for a certain medical procedure, synthetic claims can be generated that include line items for that procedure with varying patient ages, diagnoses, and treatment dates to test the MTD's handling of different scenarios. Positive test scenarios might include claims where all conditions are met, such as correct patient age and valid diagnosis codes, while negative test scenarios might involve claims with invalid diagnostic codes, incorrect patient ages, or procedures not covered under a policy. In some embodiments, positive and negative test scenarios are generated with extraneous data, intentional errors, and/or noise to further test the code for an MTD. For example, synthetic claims may be generated with additional line items that are unrelated to the procedures covered by a particular MTD, with inconsistent patient information (e.g., two claims with a different date of birth for the same patient), with typographical errors such as misspellings, or the like.

In some embodiments, test scenarios 160 may be generated with synthetic claims in the same or similar formats as submitted by medical providers. For example, each test scenario may include one or more claims formatted as electronic data interchange (EDI) files, which are commonly used by medical providers to submit claims to payers, as a Portable Document Format (PDF), or the like. By generating test scenarios in these formats, it is possible to verify the code's ability to parse and process real-world claim formats accurately. Additionally, or alternatively, test scenarios 160 may be generated with synthetic claims in the same format or structure as accepted by the code. For example, the claims may be generated as an XML file.

Test scenarios 160 are then used to test rule implementation code under test 155 (“code 155”). Test scenarios 160 may be incorporated into or used by implementation test code 150 in order to test code 155. The correct output of code 155 is known for each of test scenarios 160. Accordingly, as an output from implementation test code 150 being applied to code 155, a QA supervisor can receive a listing of all the instances in which the output from code 155 did not match the expected output for test scenarios 160. Based on the test scenarios 160 that resulted in failures, a ticket can be created in a project management platform in order to have the programmers modify code 155 to correct any identified errors. Once code 155 is performing as expected, code 155 may be pushed out via a network to one or more entities 170 (e.g., 170-1, 170-2, and 170-3), such as insurance companies, who can use code 155 to evaluate whether a medical claim should be granted or denied. Code 155 can then be implemented using the computer systems of the one or more entities 170.

Returning to MTD creation 120, MTDs 125 can be passed to AI foundational models 140. AI foundational models 140 can create and update the ML models that are used within the ecosystem of system 100, including knowledge extraction engine 115. If an error or hallucination in test scenarios or pseudocode was created, feedback provided by the QA supervisor can be used to update one or more of AI foundational models 140, which in turn helps improve accuracy of future creations of test scenarios and/or pseudocode.

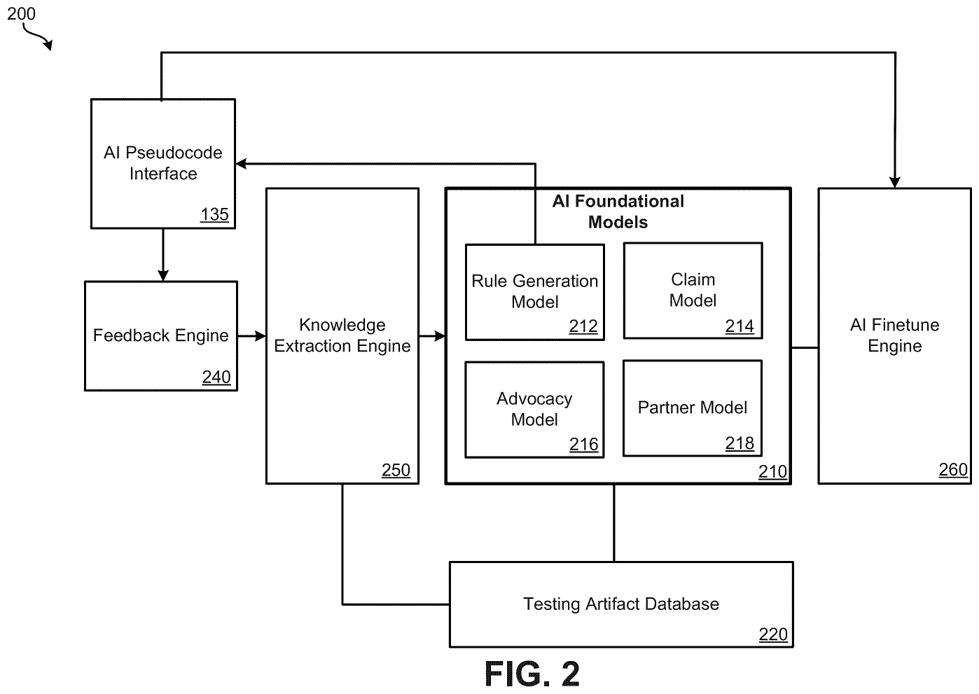

FIG. 2 illustrates a block diagram of an embodiment of a system 200 that uses artificial intelligence accessible via an application plug-in to generate pseudocode and test scenarios for quality assurance testing. System 200 can include: AI foundational models 210; testing artifact database 220; AI pseudocode interface 135; feedback engine 240; knowledge extraction engine 250; and AI finetune engine 260. Each of these components can be implemented on a computer system using purpose-designed software.

Feedback engine 240 may include one or more components that collect feedback and/or other knowledge to be used to update knowledge extraction engine 250 and/or AI foundational models 210. Feedback can, for example, include access to a new piece of knowledge or an update to an existing piece of knowledge. Additionally, or alternatively, feedback can include corrections to data retrieved by knowledge extraction engine 250 and/or generated by AI foundational models 210, as described further herein. Feedback engine 240 may include one or more interfaces to receive and process feedback for input to knowledge extraction engine 250. For example, feedback engine 240 may include one or more processes configured to receive feedback via AI-based test pseudocode interface 135. Additionally, or alternatively, feedback engine 240 may include one or more processes configured to actively retrieve and/or preprocess new and/or updated materials from which knowledge extraction engine 250 and/or AI foundational models 210 will generate new data, such as pseudocode and/or test scenarios. For example, and as described above, feedback engine 240 may include one or more processes configured to scan one or more websites and/or webpages for new medical regulations or updates to existing medical regulations.

Knowledge extraction engine 250 may include one or more components configured to generate and maintain a corpus of knowledge within testing artifact database 220. As described further herein, the corpus of knowledge within testing artifact database 220 may represent source information from medical regulations, definitions for medical procedures, definitions for medical diagnoses, insurance policies, MTDs, historical claims, billing standards, medical guidelines, manuals, or the like. Knowledge extraction engine 250 may generate and store representations of the source information in a unified manner and/or structure within testing artifact database 220 to facilitate efficient and accurate retrieval of relevant information, on which AI foundational models 210 may be trained and/or executed. Knowledge extraction engine 250 may include one or more components configured to retrieve the relevant information from the corpus of knowledge within testing artifact database 220 for AI foundational models 210 depending on the intended function to be performed by AI foundational models 210.

AI foundational models 210 may include a large-scale, pre-trained neural network, such as a large language model or the like, designed to serve as a versatile base for various downstream tasks, such as natural language processing, machine translation, generative models, predictive analytics, knowledge graphs, or the like. AI foundational models 210 may be trained on the entire corpus of knowledge generated and maintained by knowledge extraction engine 250, enabling it to learn general representations that can be fine-tuned for specific applications. AI foundational models 210 may be pre-trained on the corpus of knowledge using unsupervised, semi-supervised, supervised, and/or self-supervised learning techniques. This pre-training phase may be followed by fine-tuning on smaller, task-specific datasets to adapt the model to particular use cases, improving its performance on those tasks.

As illustrated, AI foundational models 210 may include multiple specialized models, or adaptors, specifically designed to perform particular tasks, such as rule generation model 212, claim model 214, advocacy model 216, and partner model 218. AI finetune engine 260 may fine tune each specialized model, or adaptor, to leverage the foundational knowledge encoded within AI foundational models 210, ensuring high accuracy and efficiency in their respective tasks. For example, rule generation model 212 may be optimized to create rules for MTDs based on updated medical regulations and the knowledge extracted from rules in historical MTDs. As another example, the claim model 214 may be optimized to generate synthetic claim data designed to test the rules defined in an MTD based on the knowledge extracted from application of similar rules in historical claim data. In yet another example, advocacy model 216 may be designed to determine prices for a particular medical procedure and/or service based on knowledge extracted from historical claim data related to the billed and/or approved amounts for the same or similar procedures. In another example, partner model 218 may be optimized to perform processing related to a particular entity, such as an insurance provider, based on special knowledge extracted from data specific to the particular entity.

When a large language model (LLM) on which AI foundational models 210 are based is initially applied to a new knowledge area, the model needs to update in order to function properly. Rather than updating the entire LLM, AI finetune engine 260 allows for an additional layer of processing to be performed on the output of each of AI foundational models 210 rather than updating the LLM on which the models were created.

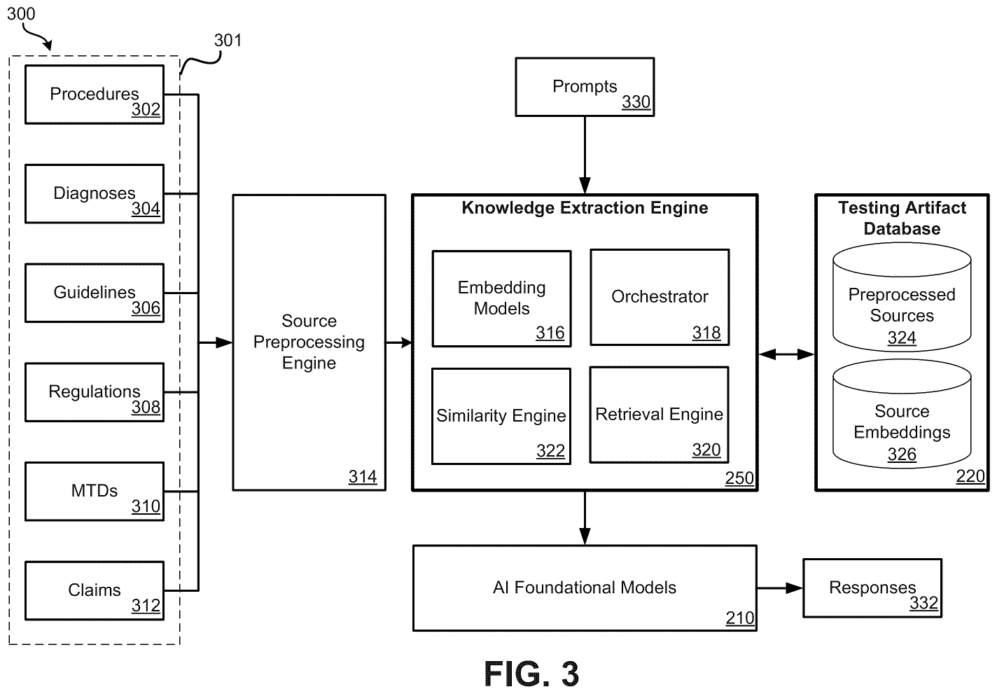

FIG. 3 illustrates an exemplary flow of data in an embodiment of a system 300 that uses retrieval augmented artificial intelligence to generate pseudocode and test data for quality assurance testing. As illustrated, system 300 can include one or more components of system 200 described above, including: knowledge extraction engine 250; testing artifact database 220; and AI foundational models 210. As further illustrated, system 300 includes source preprocessing engine 314.

Source preprocessing engine 314 may provide the same or similar functions and services as described above in relation to feedback engine 240. Source preprocessing engine 314 may include one or more components configured to collect, organize, and process source information 301 into a uniform structure and/or format for each type or source of information. For example, source preprocessing engine 314 may extract text documents into one or more predefined schemas or data objects defined for each type or source of information. As described above, source information 301 may include: medical procedures 302; medical diagnoses 304; clinical practice guidelines 306; medical regulations 308; MTDs 310; and synthetic or anonymized claims 312.

Medical procedures 302 may include descriptions, codes, and classifications of various medical and surgical procedures performed by healthcare providers. Medical procedures 302 may be obtained from standardized coding systems such as CPT and HCPCS codes. Source preprocessing engine 314 may process medical procedures 302 by normalizing the data, mapping it to standardized codes, and structuring the data for use by knowledge extraction engine 250. For example, source preprocessing engine 314 may organize medical procedures into a hierarchical structure based on categories and subcategories, create indexes based on procedure codes and their associated descriptions, add relevant metadata to each procedure entry, or the like.

Medical diagnoses 304 may include diagnostic codes and descriptions used to identify and classify diseases and health conditions. Medical diagnoses 304 may be obtained from standardized coding systems such as ICD and Systematized Nomenclature of Medicine-Clinical Terms (SNOMED CT). Source preprocessing engine 314 may process medical diagnoses 304 by verifying the accuracy of the codes, normalizing the data, and structuring it to be compatible with knowledge extraction engine 250.

Clinical practice guidelines 306 may include evidence-based recommendations and protocols for the diagnosis and treatment of various medical conditions. Clinical practice guidelines 306 may be obtained from authoritative sources such as medical associations, government health agencies, and professional societies. Source preprocessing engine 314 may process clinical practice guidelines 306 by extracting relevant information, standardizing the format, and organizing it for efficient retrieval and analysis by knowledge extraction engine 250.

Medical regulations 308 may include laws, rules, and policies governing healthcare practices, billing, and insurance coverage, as well as updates made thereto. Medical regulations 308 may be obtained from government agencies, regulatory bodies, and legal databases. Source preprocessing engine 314 may process medical regulations 308 by parsing the legal text, identifying key regulatory requirements, and structuring the information to be accessible and usable by knowledge extraction engine 250.

Claims 312 may include sample claims data that has been generated or anonymized to protect patient privacy while allowing for analysis and testing. Claims 312 may be obtained from anonymized datasets, synthetic data generation tools, and historical claims records that have been de-identified. Source preprocessing engine 314 may process claims 312 by ensuring the data is properly anonymized, structuring it in a standardized format, and preparing it for analysis and validation by knowledge extraction engine 250.

During initialization of system 300, source preprocessing engine 314 may process source information 301 in batches. Subsequently, preprocessing engine 314 may process new and/or updated source information 301 as it becomes available. Once processed, source preprocessing engine 314 may store source information 301 in preprocessed sources 324. Preprocessed sources 324 may include one or more datastores configured to store source information 301 in its original format and/or in the format produced by source preprocessing engine 314. For example, preprocessed sources 324 may include document management systems or text repositories for storing text documents, as may be the case for clinical practice guidelines 306 and/or medical regulations 308. As another example, preprocessed sources 324 may include relational databases or NoSQL databases for storing structured data, such as procedures 302, diagnoses 304, and claims 312.

As further described above, knowledge extraction engine 250 may include one or more components configured to generate, manage, and interact with a corpus of knowledge in support of the functions performed by AI foundational models 210. For example, and as illustrated, knowledge extraction engine 250 may include: embedding models 316; orchestrator 318; retrieval engine 320; and similarity engine 322. As further described herein, the components of knowledge extraction engine 250, testing artifact database 220, and AI foundational models 210 may form a neural network architecture that combines retrieval-based and generative approaches to enhance the performance of various natural language and structured data processing tasks, such as generating new or updated MTDs from new or updated medical regulations, generating code to implement MTDs, generating test scenarios including synthetic claims to test MTD code, or the like.

Embedding models 316 may include one or more components configured to convert input data, such as source information 301 from directly from source preprocessing engine 314 and/or preprocessed sources 324, as well as prompts 330, into dense vector representations in a continuous vector space, facilitating efficient similarity searches, comparisons, and downstream machine learning tasks. Embedding models 316 may include transformer-based architectures, such as a Bidirectional Encoder Representations from Transformers (BERT) model, a Robustly Optimized BERT Pre-training Approach (RoBERTa), or the like. Embedding models 316 may generate embeddings for source information 301 by processing text data through these architectures to produce contextualized embeddings that capture semantic meaning. As described herein, capturing the semantic meaning may refer to the ability of embeddings to represent the context within which words, terms, or other entities in text are used, the relationships between entities in the same source text, and/or the relationships across sources. For example, embeddings generated from clinical practice guidelines 306, medical regulations 308, and/or MTDs 310 capture various entities, such as the descriptions, codes, or names of medical procedures and medical diagnoses, as well as the relationships between those entities, such as the sequence of procedures typically performed together, the common diagnoses that necessitate certain treatments, and the regulatory conditions under which specific procedures are covered.

The ability to generate embeddings that capture the semantic meaning enable knowledge extraction engine 250 to understand and retrieve relevant information by recognizing the semantic connections within and across different types of documents. For instance, embeddings generated from clinical practice guidelines 306 may capture the recommended treatment protocols for specific medical conditions, while embeddings from medical regulations 308 might encapsulate the legal requirements and coverage policies. Similarly, embeddings from MTDs 310 could represent the criteria for claim approval, denial, or modification based on these regulations.

By capturing the semantic meaning, embedding models 316 facilitate the efficient retrieval of relevant documents or passages that share similar contexts or entities, even if the exact wording differs. This capability is crucial for tasks such as generating new or updated MTDs from new or updated medical regulations, generating code to implement MTDs, and generating test scenarios including synthetic claims to test MTD code. The embeddings serve as a foundational layer that supports these complex tasks by providing a rich, context-aware representation of the source information.

Embedding models 316 may be trained to generate embeddings by using large-scale pre-training on diverse text corpora followed by fine-tuning on domain-specific data. For example, and as described further herein, fine-tuning embedding models 316 may include training a transformer model on similar and dissimilar pairs of documents, passages, terms, and other entities to enhance its ability to discern contextual similarities and differences and subsequently generate similar embeddings for similar concepts, and dissimilar embeddings for dissimilar concepts.

The embeddings generated for source information 301 by embedding models 316 may be stored in source embeddings 326. Source embeddings 326 may include one or more datastores configured to efficiently store and manage dense vector representations of source information 301. For example, source embeddings 326 may include one or more relational databases, such as a PostgreSQL database, NoSQL databases like MongoDB, or specialized vector databases such as Facebook AI Similarity Search (FAISS) or Approximate Nearest Neighbors Oh Yeah (ANNOY), or the like. These datastores are designed to support rapid retrieval and comparison of embeddings, enabling the knowledge extraction engine 250 to perform its tasks effectively.

Embedding models 316 may store the embeddings for the various types and/or sources of source information 301 in source embeddings 326 by organizing them into separate tables, collections, or indices based on the type of source information. For instance, embeddings for medical procedures may be stored separately from embeddings for medical diagnoses, clinical practice guidelines, or medical regulations. This structured organization facilitates efficient and accurate retrieval of relevant embeddings when processing queries or performing similarity searches.

Embedding models 316 may further generate and store multiple types of embeddings for similar pieces of source information 301, as described further herein. Each type or category of embedding may represent uniquely identifiable features common to a particular type of source information. For example, uniquely identifiable features of medical regulation documents may include: the title of the document; the organization of information within the document (e.g., based on the headings and subheadings within the document); the text on each individual page or within each section of the document, or the like. As another example, uniquely identifiable features for a medical procedure or diagnosis may include: one or more standardized codes for procedure or diagnosis; a plain English description or definition of the procedure or diagnosis; a shorthand description of the procedure or diagnosis commonly found in clinical notes; or the like.

Links may be created between embeddings in source embeddings 326 and the corresponding piece of information in preprocessed sources 324. For example, a link or other reference locator for a text document in preprocessed sources 324 may be included in the corresponding entry of the embedding in source embeddings 326. Additionally, or alternatively, text documents in preprocessed sources 324 may be indexed by their corresponding embedding in source embeddings 326.

Retrieval engine 320 may include one or more components configured to retrieve embeddings from source embeddings 326 that may be relevant to prompts 330 and/or the generation of responses 332 by AI foundational models 210. For example, retrieval engine 320 may include search algorithms and indexing systems that efficiently query the embedding space. Retrieval engine 320 may retrieve relevant embeddings from a collection of embeddings in source embeddings 326 by performing similarity searches within the collection based on the embedding of the input prompt, a previously retrieved embedding from a different collection of embeddings, and/or an embedding of one or more supporting documents provided with the input prompt, as described further herein. Retrieval engine 320 may retrieve embeddings in response to input from orchestrator 318, which may specify the collection of embeddings from which to retrieve the embeddings, and/or specify the criteria and parameters for the search. Retrieval engine 320 may provide the retrieved embeddings to similarity engine 322.

Similarity engine 322 may include one or more components configured to evaluate the relevance of the information retrieved by retrieval engine 320 and/or rank them according to their importance or relevance to prompts 330 or other information retrieved by retrieval engine 320. For example, similarity engine 322 may include ranking algorithms and relevance scoring mechanisms. Similarity engine 322 may determine the relevance of the information retrieved by retrieval engine 320 by comparing the embeddings of the retrieved documents with the embeddings of the prompts, using metrics such as cosine similarity or Euclidean distance. This ranking process ensures that the most relevant information is prioritized for further processing by knowledge extraction engine 250 and/or AI foundational models 210.

Orchestrator 318 may include one or more components configured to coordinate execution of the remaining components of knowledge extraction engine 250 and interface with AI foundational models 210 in response to prompts 330. For example, orchestrator 318 may include a query processor configured to determine which type, or types, of source information 301 of embeddings in source embeddings 326 are relevant to a given prompt and/or will help improve the response generated by AI foundational models 210. For example, given a prompt with instructions to generate a new MTD from a medical regulation, the query processor may determine that similar medical regulations, as well as MTDs generated therefrom, should be retrieved to provide additional context for AI foundational models 210. As another example, given a prompt with instructions to generate synthetic claims based on an MTD, the query processor may determine that similar MTDs, as well as synthetic claims used to test those MTDs, should be retrieved to provide examples for AI foundational models 210.

The query processor may determine which type, or types, of source information 301 are relevant based on the prompt itself and/or additional information provided with the prompt. For example, given an embedding of a prompt from embedding models 316, orchestrator 318 may apply a classifier model trained to determine the type of request being submitted. As described above, the types of requests may include requests to generate MTDs from regulations, generate code from MTDs, generate synthetic claims from MTDs, or the like. The classifier model may be trained on a dataset of labeled prompts to recognize patterns and keywords that correspond to different types of requests. Based on the type of request, orchestrator may use a predefined mapping to determine the types of source information 301 that are relevant.

Additionally, or alternatively, orchestrator 318 may determine the types of source information 301 that are relevant based on the source of the prompt. For example, knowledge extraction engine 250 may have a plurality of interfaces through which prompts may be submitted (e.g., corresponding to different user interface tools, or the like). Based on the interface through which a prompt is received, orchestrator 318 may use a predefined mapping to determine the types of source information 301 that are relevant.

Orchestrator 318 may include a task scheduler component that implements various logic to control the order in which relevant source information 301 is retrieved. For example, after determining that relevant medical regulations should be retrieved, orchestrator 318 may sequentially task retrieval engine 320 and similarity engine 322 with retrieving embeddings for relevant source documents, followed by embeddings for relevant sections within a subset of the relevant source documents, followed by embeddings for the text within a subset of the relevant sections.

Orchestrator 318 may include an output component that interfaces with AI foundation models 210. The output component may augment the embedding of the original prompt with the embeddings for the relevant information retrieved by retrieval engine 320. Augmenting the embedding of the original prompt with the embeddings for the relevant information may include concatenating the embeddings, averaging them, or applying more advanced techniques such as attention mechanisms to weigh the importance of each retrieved embedding based on its relevance to the prompt. This augmented embedding provides a richer, context-aware representation that can be used by AI foundational models 210 to generate more accurate and contextually appropriate responses. For example, if the prompt is to generate a new MTD from a medical regulation, the output component may combine the embedding of the prompt with embeddings of similar medical regulations and existing MTDs. This combined embedding is then fed into AI foundational models 210, which use the enriched context to produce a detailed and compliant MTD. Similarly, if the prompt is to generate synthetic claims based on an MTD, the output component may integrate embeddings of similar MTDs and historical synthetic claims, allowing the AI foundational models 210 to generate realistic claims.

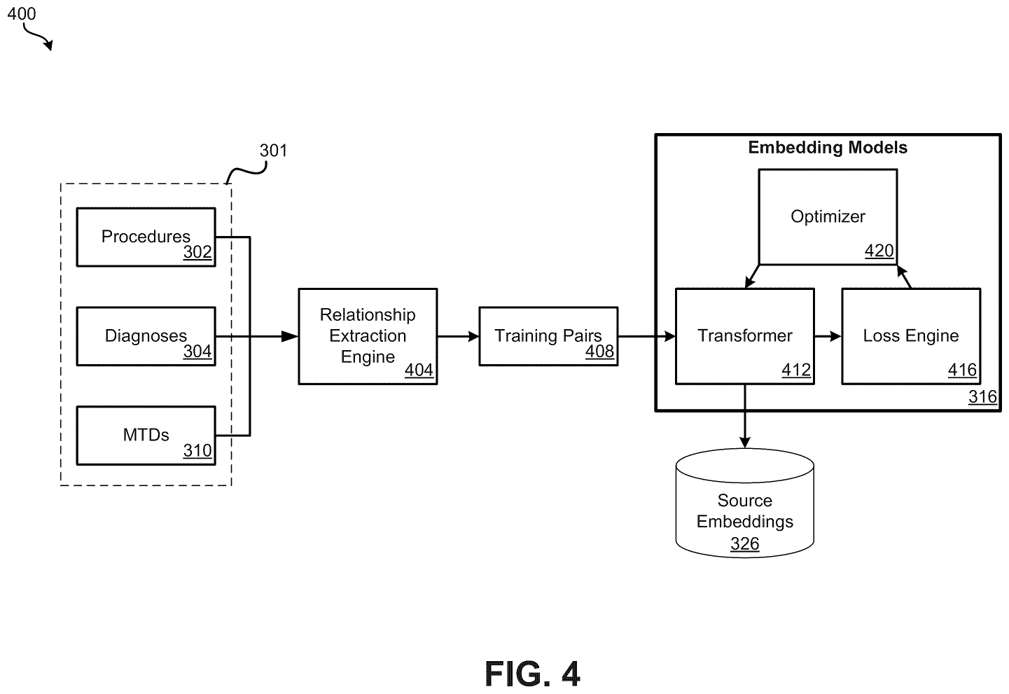

FIG. 4 illustrates flow diagram for training a system 400 to generate contextual embeddings for source information. As illustrated, system 400 can include one or more components of system 300 described above, including embedding models 316 and source embeddings 326. As further illustrated, system 400 includes relationship extraction engine 404.

Relationship extraction engine 404 may provide the same or similar functions and services as described above in relation to source preprocessing engine 314. Relationship extraction engine 404 may include one or more components configured to generate training pairs 408 from source information 301. Training pairs 408 may include pairs of similar and dissimilar source information 301. Each pair of training pairs 408 may be labeled to indicate whether the pair is similar or dissimilar. As described herein, similar pairs of source information include pairs of information (e.g., documents, passages, terms, definitions, and/or other entities) that are contextually or semantically related, such as documents or data entries that share common themes, topics, or entities. On the other hand, dissimilar pairs may include pairs of information that are contextually or semantically unrelated.

Contextually related pairs of information may be connected based on the context in which they appear and may be determined by external factors. For example, a diagnosis and the approved treatment for that diagnosis are contextually similar because of an external relationship that links the two (e.g., the approval by an entity of the treatment for that diagnosis. Semantically related pairs of information may be connected based on their meaning and content, and may be determined by their intrinsic relationship. For example, a plain English definition of a diagnosis, a clinician's shorthand notation of the diagnosis, and a uniquely identifiable code for the diagnosis are semantically similar because they all refer to the same diagnosis.

Relationship extraction engine 404 may generate training pairs 408 from similar and dissimilar pairs of source information 301 using various techniques and algorithms designed to identify and label these relationships. Relationship extraction engine 404 may apply predefined rules and heuristics to identify relationships based on specific criteria. For example, rules might be established to match medical procedure codes with their corresponding descriptions. As another example, rules might be established to match codes for procedures 302 with MTDs 308 that include the procedure code. As another example, rules might be established to match MTDs 310 with regulations cited therein. In yet another example, rules might be established to match diagnoses 304 with approved procedures 302, or vice versa, within regulations and/or MTDs. In some embodiments, relationship extraction engine 404 allows for manual annotation. For example, domain experts may manually review and label pairs of source information 301 and provide them to relationship extraction engine 404 to codify the relationship.

As further described above, embedding models 316 may include one or more components configured to convert input data, such as source information 301 from directly from source preprocessing engine 314 and/or preprocessed sources 324, as well as prompts 330, into dense vector representations in a continuous vector space. For example, and as illustrated, embedding models may include: transformer 412; loss engine 416; and optimizer 420.

Transformer 412 may include a multi-layered architecture designed to process and encode input text into dense vector representations. The multi-layered architecture may include an encoder and decoder, with the encoder responsible for transforming the input data into a set of hidden states and the decoder generating the final embeddings from these states. Transformer 412 may utilize self-attention mechanisms to capture long-range dependencies and contextual relationships within the input text, ensuring that the generated embeddings accurately represent the semantic and contextual information. As described further herein, training pairs 408 may be used to train transformer 412 to generate embeddings that accurately represent the semantic and contextual information of the input data.

Loss engine 416 may include components configured to calculate one or more loss functions during the training process of transformer 412. The loss function may measure the difference between the model's predicted outputs and the actual target values, guiding the optimization process. For example, given a pair of similar inputs in training pairs 408, loss engine 416 may calculate a similarity loss that measures how close the embeddings of the similar pairs are in the vector space. Conversely, for dissimilar pairs, the loss engine 416 may calculate a dissimilarity loss that encourages the embeddings to be further apart. The loss functions used by loss engine 416 may include contrastive loss or triplet loss, which help in maximizing the similarity for similar pairs and minimizing it for dissimilar pairs.

Optimizer 420 may include algorithms designed to update the parameters in transformer 412 based on the loss calculated by loss engine 416. For example, optimizer 420 may use stochastic gradient descent (SGD), Adaptive Moment Estimation (ADAM), Root Mean Square Propagation (RMSprop), or the like to adjust the weights and biases of transformer 412 during training. Optimizer 420 ensures that the model converges to an optimal set of parameters that produce high-quality embeddings for various types of input data.

As described above, training pairs 408 may be used to train transformer 412 to recognize and differentiate between contextually and/or semantically similar and dissimilar information. This capability is crucial for tasks such as semantic search, information retrieval, and the generation of context-aware embeddings. Using training pairs 408, transformer 412 can learn to generate embeddings for new information and prompts within the embedding space that are close in proximity to previous embeddings for contextually and/or semantically related source information. This in turn enables knowledge extraction engine 250 to retrieve and provide the correct knowledge to AI foundational models 210, which enables AI foundational models 210 to minimize hallucinations in the responses it generates, cite to the correct source information, and produce structured data responses (e.g., synthetic claims) from unstructured data inputs (e.g., medical regulations and MTDs).

To further illustrate the technical improvements provided by supplementing AI foundational models 210 with embedding models 316 as described herein, consider a conventional large language model trained on a corpus of medical regulations to generate MTDs. Given an updated or new medical regulation alone, the conventional large language model may produce responses that are contextually relevant but lack precise alignment with existing MTDs and detailed regulatory requirements. This is because the model relies solely on its pre-trained knowledge and may not have the capability to access or integrate the most up-to-date contextual information.

Furthermore, without the support of embedding models, the conventional model may struggle to accurately interpret the nuances and specific changes in the new medical regulation, potentially leading to inaccuracies or omissions in the generated MTDs. For instance, the model might miss subtle shifts in regulatory language or fail to recognize the implications of new procedural guidelines, resulting in MTDs that do not fully comply with the latest standards.

In contrast, when AI foundational models 210 are supplemented with embedding models 316, the system leverages dense vector representations that capture the semantic and contextual relationships within the data. Embedding models 316, trained using training pairs 408, provide accurate and context-aware embeddings of both new and existing medical regulations. This allows knowledge extraction engine 250 to retrieve the most relevant information and present it to AI foundational models 210, ensuring that the generated MTDs are precise, up-to-date, and fully compliant with the latest regulations.

As another example, consider a conventional large language model trained on a corpus of approved and denied claims based on the application of one or more MTDs. When given a new or updated MTD and instructions to generate structured claim data for multiple claims that both satisfy and fail to satisfy the requirements of the new or updated MTD, the conventional large language model may produce claims that are inconsistently aligned with the updated criteria. This inconsistency arises because the model may not fully understand or accurately incorporate the specific changes and detailed requirements introduced by the new or updated MTD.

Furthermore, when the conventional model attempts to generate a valid or invalid claim for a procedure described in text form in the MTD, it may fail to populate the claim with the correct code that corresponds to the procedure's name or description. This is due to the model's limited ability to understand and accurately map the textual description of the procedure to its corresponding standardized code. Similarly, when attempting to generate a valid claim for a procedure justified by a particular diagnosis, the conventional model may fail to populate the claim with the correct code that corresponds to the diagnosis. In both cases, the claims generated by the conventional model would be unusable to accurately test code designed to implement the MTD.

In contrast, by supplementing the large language model with embedding models that generate context-aware embeddings, the system gains the ability to accurately capture and integrate the nuanced details and specific criteria outlined in the new or updated MTD. Embedding models, trained using training pairs that include similar and dissimilar relationships between procedures, diagnoses, and their corresponding codes, provide dense vector representations that reflect the precise semantic and contextual details of the MTD. These context-aware embeddings enable the knowledge extraction engine to efficiently retrieve and present relevant and up-to-date information to AI foundational models. This enriched context allows the AI foundational models to generate structured claims that are both consistent with the latest MTD and accurately reflect the specific approval and denial criteria. Furthermore, the system can correctly map textual descriptions of procedures and diagnoses to their standardized codes, ensuring that each claim includes the intended CPT and ICD codes for testing purposes.

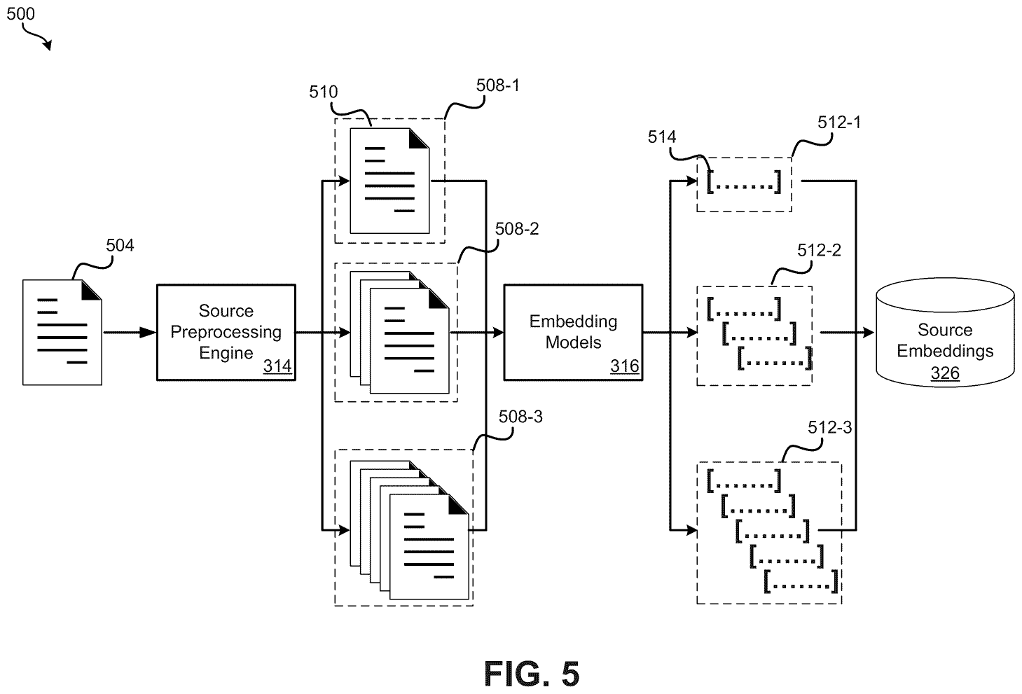

FIG. 5 illustrates an exemplary flow of data within a system 500 when generating embeddings of source documents. As illustrated, system 500 can include one or more components of system 300 described above, including: source preprocessing engine 314; embedding models 316; and source embeddings 326. As described above, source preprocessing engine 314 may receive various types of information during initialization and/or run-time and convert them into a predefined format and/or structure. For example, and as illustrated, source preprocessing engine 314 may receive document 504. As described herein, document 504 may represent a text document, such as a medical regulation document, a clinical practice guideline, or the like.

As further illustrated, source preprocessing engine 314 may process document 504 into multiple classes 508 of features 510. Each class of features may correspond to uniquely identifiable components or pieces that are common to documents or objects of the same type, from the same source, or the like. For example, assuming document 504 is a text document, the multiple classes 508 may include: a first class 508-1 corresponding to the title of the document; a second class 508-2 corresponding to the organizational components within document 504 (e.g., headings and subheadings); and a third class 508-3 corresponding to portions of text within document 504 (e.g., the text on each page, within each section, or the like). Additional and/or alternative classes of features may be defined for different categories or types of information. For example, medical procedures and/or diagnoses may be processed into classes such as: a first class corresponding to standardized codes (e.g., CPT codes for procedures, ICD codes for diagnoses); a second class corresponding to plain English descriptions or definitions of the procedures or diagnoses; a third class corresponding to shorthand descriptions or notations commonly found in clinical notes; and a fourth class corresponding to billing information associated with the procedures or diagnoses.

For each class of features, source preprocessing engine 314 may extract the features into a common format and/or structure. This may involve converting text into a standardized schema, mapping terminology to standardized codes, and organizing information into hierarchical structures that reflect the hierarchical relationships in the data. For instance, for a class corresponding to standardized codes, source preprocessing engine 314 may map medical procedure descriptions to their corresponding CPT codes and diagnosis descriptions to their corresponding ICD codes. This ensures that all procedural and diagnostic information is uniformly represented, facilitating accurate retrieval and analysis. In the case of plain English descriptions or definitions, source preprocessing engine 314 may normalize the text to remove variations and inconsistencies. For claim data, source preprocessing engine 314 may extract and structure the data into predefined fields such as procedure codes, diagnosis codes, and associated costs. For hierarchical information, such as the organization of headings and subheadings within a document, source preprocessing engine 314 may extract features that include the underlying information (e.g., the title of a heading or subheading) and the relationship to other information (e.g., the title of the parent heading).

The features within each class, such as the title of document 504, the headings/subheadings, sections of text, or the like, may be provided to embedding models 316 to generate corresponding categories 512 of embeddings 514. Each category 512 of embeddings 514 may correspond to a class 508 of features 510. For example, categories 512 may include: first category 512-1 corresponding to first class 508-1; second category 512-2 corresponding to second class 508-2; and third category 512-3 corresponding to third class 508-3. Embedding models 316 may generate embeddings 514 within each category 512 corresponding to each feature within each class 508. For example, document 504 may result in embedding models 316 generating: an embedding of the title of document 504; embeddings corresponding to each heading/subheading, as well as their internal relationships; and embeddings corresponding to the text on each page of document 504.

Once generated, embeddings 514 may be stored in source embeddings 326. Each category 512 of embeddings 514 may be stored separately. For example, source embeddings 326 may include separate tables, collections, indices, or the like corresponding to each category 512. For instance, embeddings for first category 512-1 may be stored in one table, while embeddings for second category 512-2 may be stored in another.

Generating separate embeddings for uniquely identifiable features within the same piece of source information may address technical challenges associated with ensuring detailed and context-aware representations of complex data, such as medical regulations, MTDs, claims, or the like. To illustrate, consider conventional transformer models. While such models may be used to capture long-range dependencies and contextual relationships within input text, their ability to do so degrades as the length of the input text increases. In some cases, this degradation may be due to the quadratic complexity of the self-attention mechanism, which struggles with the increased computational and memory demands of processing very long sequences of text. As a result, inputting the entire text of lengthy and complex documents, such as medical regulations, may result in an embedding that loses critical details and fails to accurately represent the intricate relationships within the text.

By generating separate embeddings for distinct features within the same document—such as titles, headings, subheadings, and individual sections of text—each embedding can focus on a manageable portion of the document. This approach ensures that the embeddings capture the specific context and content of each feature without being overwhelmed by the entire document's length. These feature-specific embeddings can then be aggregated or used in conjunction to provide a comprehensive and nuanced representation of the entire document.

This method not only mitigates the limitations of transformer models when dealing with long texts but also enhances the accuracy and relevance of the embeddings. By preserving the granular details and contextual relationships of each part of the document, the system can perform more precise similarity searches, information retrieval, and downstream tasks such as generating new MTDs, validating claims, and ensuring regulatory compliance.

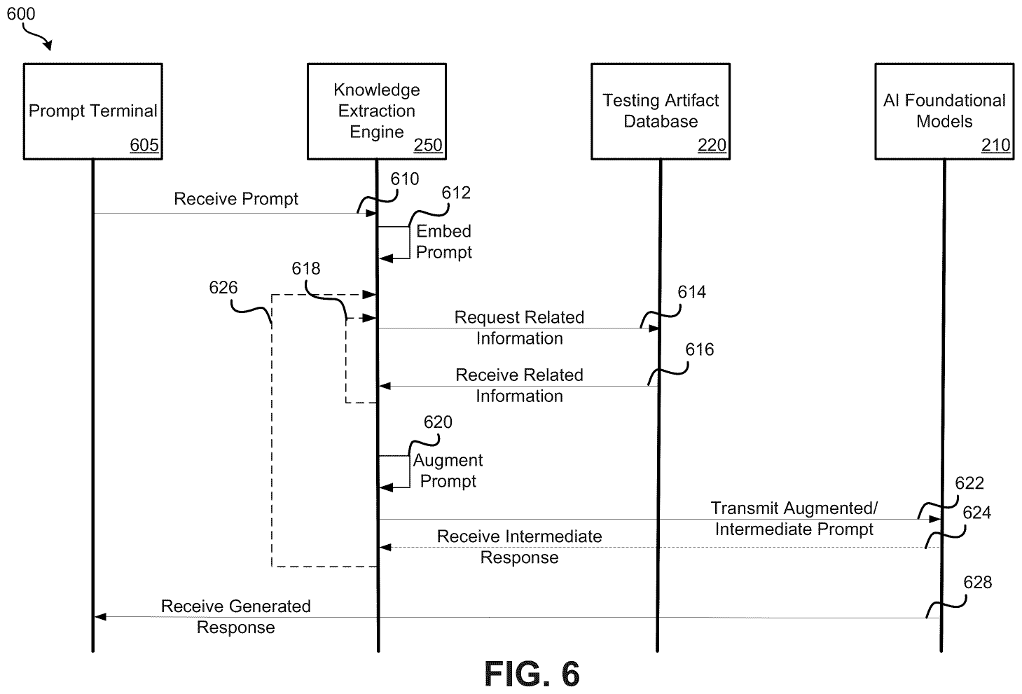

FIG. 6 illustrates a sequence diagram 600 that depicts interactions between components of a retrieval augmented generation system in response to a prompt. As illustrated, the components of the system may include one or more components of system 200, as described above, including: knowledge extraction engine 250; testing artifact database 220; and AI foundational models 210. As further illustrated, the system may include prompt terminal 605. Prompt terminal 605 may represent one or more physical computing devices and/or software applications configured to provide one or more interfaces for interacting with the other components of the system, such as combined interface 130 and/or AI-based test pseudocode interface 135, as described further herein. For example, prompt terminal 605 may provide one or more interfaces that enable a user to enter and submit a prompt to AI foundational models 210, such as to generate a new or updated MTD, and review the response.

As illustrated, the interactions may begin with knowledge extraction engine 250 receiving a prompt at step 610. The prompt may include plain text data describing the desired response from AI foundational models 210. Additionally, or alternatively, the prompt may include one or more pieces of supporting information, such as a medical regulation, a medical technical document, or the like. At step 612, knowledge extraction engine 250 (e.g., embedding models 316) may generate one or more embeddings from the prompt.

At step 614, knowledge extraction engine 250 may request related information from testing artifact database 220. For example, in response to a prompt requesting the generation of a new and/or updated MTD from a new medical regulation, orchestrator 318 may determine that embeddings for similar medical regulations and associated existing MTDs should be retrieved and task retrieval engine 320 with retrieving them from source embeddings 326. As described further above, the related information may be retrieved using the one or more embeddings of the prompt. For example, retrieval engine 320 may search within source embeddings 326 for embeddings of medical regulations that are within close proximity in the embedding space to the embedding of the medical regulation provided with the prompt.

At step 616, knowledge extraction engine 250 may receive the related information from testing artifact database 220. After receiving the related information, knowledge extraction engine 250 may identify the most relevant information received from testing artifact database 220. For example, similarity engine 322 may compare the one or more embeddings of the prompt with the retrieved embeddings from source embeddings 326 to identify the most similar embedding.

As illustrated by the dashed line, knowledge extraction engine 250 may perform additional iterations of step 614 and step 616 at step 618 until all of the relevant information has been retrieved. To illustrate, and continuing with the example above, a first iteration may be performed to identify the most relevant medical regulation documents. Subsequent iterations may then be performed to identify the most relevant sections within those documents. The embeddings for these sections may then be used to confirm that the documents are relevant and/or identify the sections that are most relevant within the documents. Further iterations may then be performed to identify the most relevant portions of text within the document.

Similar rounds of iterations may be used to identify the most relevant MTDs, either based on the embedding of the prompt, or in combination with the most the embeddings of the most relevant medical regulations. For example, retrieval engine 320 may retrieve embeddings for MTDs that are close in proximity to the one or more embeddings of the most relevant medical regulation retrieved in prior iterations. Additionally, or alternatively, retrieval engine 320 may identify one or more MTDs from a mapping of MTDs to the medical regulations from which they are generated.

At step 620, knowledge extraction engine 250 may augment the initial prompt with the related information. For example, and as described above, orchestrator 318 may concatenate the one or more embeddings of the prompt with the one or more embeddings of the related information. Additionally, or alternatively, orchestrator 318 may retrieve the source information (e.g., from preprocessed sources 324) for the related information based on their respective embeddings and concatenate it with the information from the prompt.

At step 622, knowledge extraction engine 250 may transmit the augmented prompt to AI foundational models 210 for completion of the actions requested by the prompt. For example, AI foundational models 210 may generate the requested content (e.g., a new or updated MTD, synthetic claims, software code, etc). Finally, at step 620, the results from AI foundational models 210 may be received by prompt terminal 605.

As further illustrated by the dashed lines, the interactions may optionally include step 624, in which knowledge extraction engine 250 receives an intermediate response from AI foundational models 210. In some embodiments, prompts implicitly or explicitly include compound requests, that call for completion of different tasks by different task-specific models of AI foundational models 210. For example, a request to generate a new or updated MTD from an updated medical regulation may implicitly include a request for a first task associated with identifying the one or more differences between existing medical regulations and the updated medical regulation, followed by a second task modifying an existing MTD to incorporate the one or more differences. As another example, a prompt may request both the generation of a new or updated MTD as well as synthetic claims that can be used to test the new MTD. In these cases, knowledge extraction engine 250 may transmit an intermediate request at step 622 and receive an intermediate response at step 624.

Based on the intermediate response, knowledge extraction engine 250 may perform another iteration of step 614 through step 622 at step 628. For example, upon receiving a new or updated MTD at step 624, knowledge extraction engine 250 may retrieve historical claims that are related to the new or updated MTD at step 616. Subsequently, the historical claims may be transmitted to AI foundational models 210 with the new or updated MTD for generation of synthetic claims.

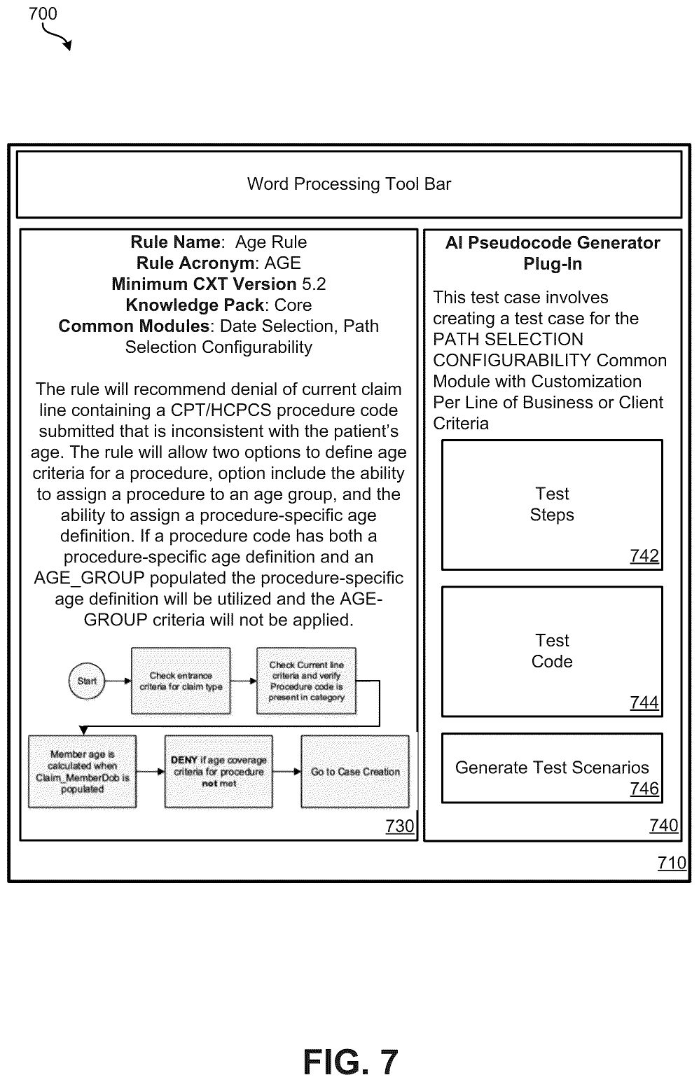

FIG. 7 illustrates an embodiment 700 of a plug-in implemented in a word processing application 710 to generate pseudocode and code for quality assurance testing. Within word processing application 710, an MTD can be loaded, such as the MTD presented in window 730. This MTD may be created as described above in relation to MTD 125 of FIG. 1. For example, a separate interface may be presented through which a new or updated medical regulation may be uploaded, or otherwise supplied, to generate a new or updated MTD. Additionally, or alternatively, the MTD may be automatically generated in response to a new or updated medical regulation being detected on a source system, at which point the generated MTD can be displayed for review and approval in window 730 along with the underlying medical regulations from which it was generated.

Presented concurrently with window 730 is AI pseudocode generator plug-in 740, which may be launched via word processing tool bar 720. Based on the content of the MTD in window 730, plug-in 740, via knowledge extraction engine 250 and/or AI foundational models 210 (which can be executed at a remote server), can generate test steps 742 and/or test code 744. Test steps 742 can represent the pseudocode that the QA administrator is to code and/or review. Test steps 742 can include a sequence of steps that need to be performed when analyzing a claim for compliance with an approved MTD. Test code 744 can represent a proposed implementation of the MTD and/or test steps 742 in one or more software languages.

Plug-in 740 may further include test scenario interface 746. In response to interaction with test scenario interface 746, plug-in 740, via knowledge extraction engine 250 and/or AI foundational models 210, can generate test scenarios, including synthetic claim data, that can be used to test implementations of the MTD, such as test code 744. As described further above, such test scenarios may be generated based on the content of the MTD in window 730, test steps 742, and/or test code 744.

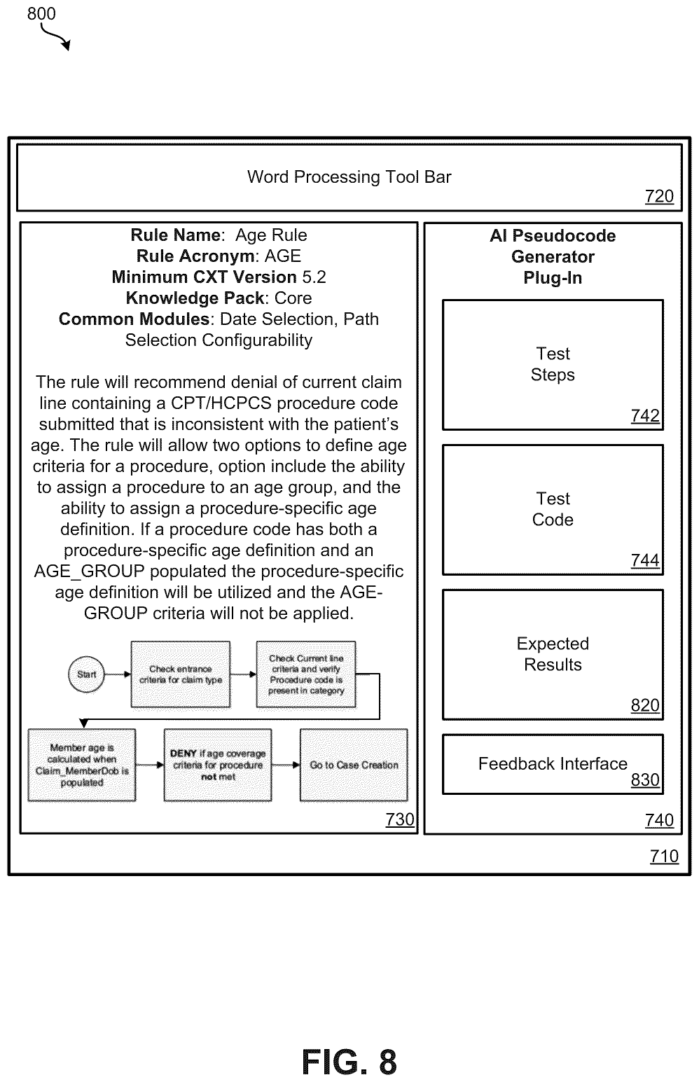

FIG. 8 illustrates an embodiment 800 of a plug-in implemented in a word processing program to solicit feedback based on generated pseudocode and code for quality assurance testing. In addition to presenting test steps 742 and/or test code 744, AI Pseudocode generator plug-in 740 presents expected results 820 and feedback interface 830. Expected results 820 can indicate the results that are expected to be generated from applying test steps 742 and/or test code 744 to the test scenarios generated for the MTD. For example, expected results 820 may include a list of the test scenarios and whether they comply with the MTD. Additionally, or alternatively, expected results 820 may indicate, for each test step, the value of the underlying data within the test scenario being analyzed and the expected result of the analysis.

Feedback interface 830 may allow a QA administrator to provide feedback on both test steps 742, test code 744, and expected results 820. In some embodiments, feedback interface 830 may be simple in that the QA administrator selects either “thumbs up” or “thumbs down” feedback. Additionally or alternatively, the QA administrator could add long-form feedback, such as in the form of one or more sentences. Such feedback could be obtained by feedback engine 240 to improve knowledge extraction engine 250 and/or AI finetune engine 260 to improve AI foundational models 210.

While illustrated and described in relation to plug-ins implemented in a word processing program, the capabilities described herein may be implemented in the form of other interfaces and/or application integrations. For example, the capabilities described above in relation to generating MTDs, pseudocode, code, test scenarios, or the like, can be implemented in the form of a standalone application. As another example, such capabilities may be accessible via one or more internet browser plug-ins. In yet another example, such capabilities may be accessible via a chat interface presented by a standalone application executing on a local machine and/or a web application provided via one or more web browsers.

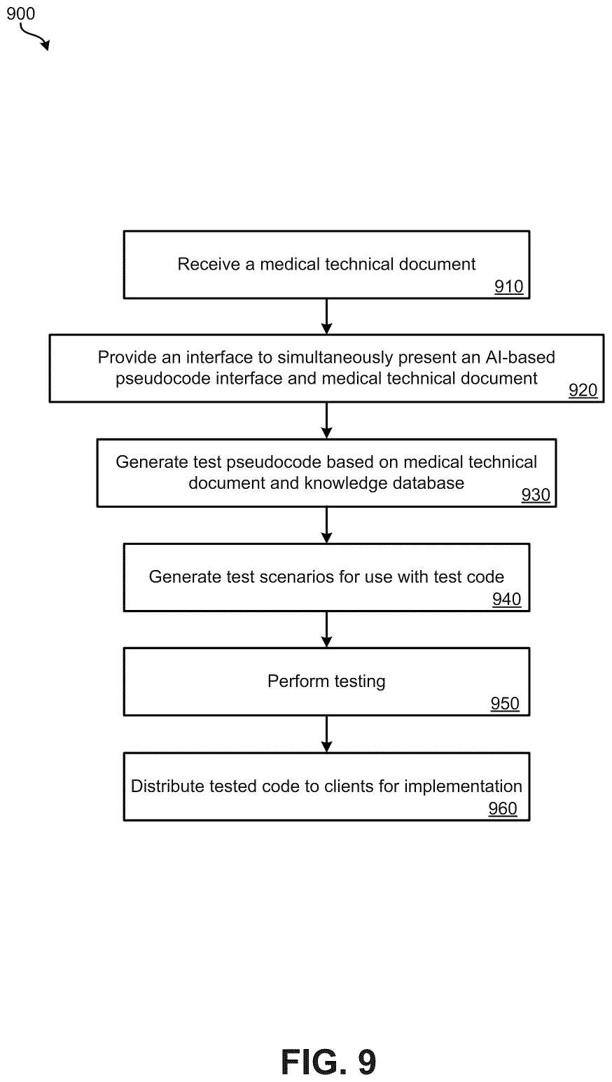

Various methods may be performed using the arrangements of FIGS. 1-4. FIG. 9 illustrates an embodiment of a method for generating pseudocode for quality assurance testing. Method 900 can be performed using the arrangements of FIGS. 1-4. Method 900 can be performed using one or more computing systems.

At block 910, a medical technical document (MTD) is received. The MTD may be electronically received, such as in the form of a document file. The MTD may have been previously created based on one or more regulations, such as detailed in relation to FIG. 1.

At block 920, an interface can be provided to simultaneously present an AI-based pseudocode interface and an MTD. The interface may be provided by a plug-in being loaded in a word processing program. Alternatively, a dedicated application can be used that allows for simultaneously presentation of an AI-based pseudocode interface and an MTD. Whether a plug-in a word processing program is used or a dedicated application, the interface may resemble, for example, the arrangement of FIG. 7.

At block 930, test pseudocode is generated based on the MTD and a stored knowledge database. The test pseudocode created can include test steps and the expected output of such test steps. The stored knowledge database can store hundreds or thousands of other instances of MTDs and description corresponding to test pseudocode that is used to test code generated based on the MTD.

At block 940, test scenarios are generated for use with the code under test. The test scenarios can be created by a programmer (e.g., QA supervisor) or directly by in place of the test pseudocode by a computer system. Each test scenario can be applied to the code under test to determine if the proper outcome is generated, such as approval or rejection of a claim.

At block 950, testing is performed using the scenarios of block 940 and the code under test. This testing can be performed by the computer system in an automated fashion such that the QA administrator is notified of instances where the expected output did not match the actual output of a test scenario.

At block 960, the tested code is distributed to one or more clients for implementation. The code that was previously tested can now be used to perform review, analysis, and approval of claims.

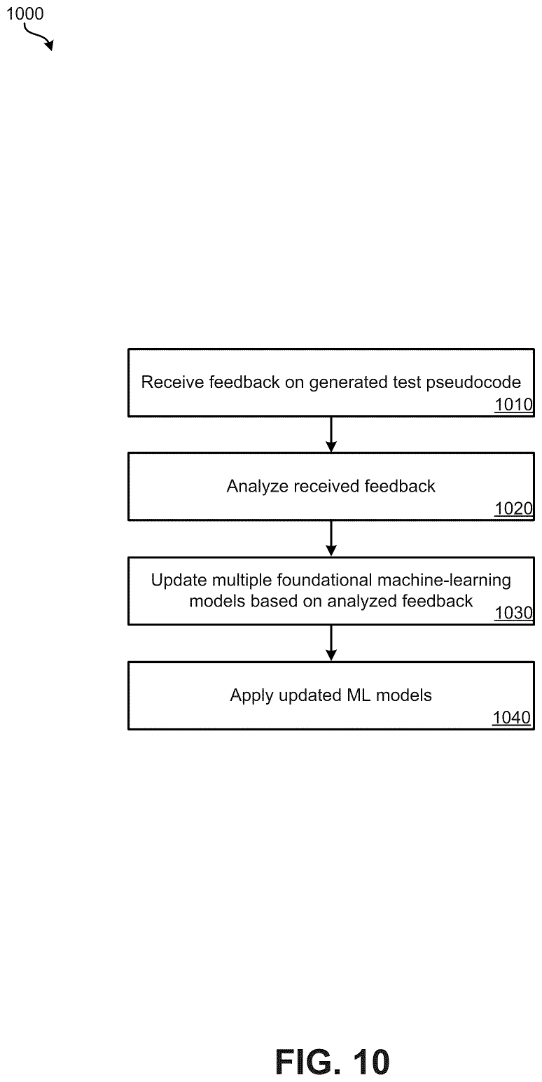

FIG. 10 illustrates an embodiment of a method for implementing code created based on generated pseudocode for quality assurance testing. Method 1000 can be performed using the arrangements of FIGS. 1-4. Method 1000 can be performed using one or more computing systems.

At block 1010, feedback is received. In response to reviewing pseudocode test steps and expected results, a QA supervisor can submit feedback. This feedback may be in the form of corrections, comments, a score, approval/disapproval (e.g., thumbs up, thumbs down), etc.

At block 1020, the feedback is analyzed. The feedback can be analyzed by a feedback engine, such a feedback engine 240. The analyzed feedback can be further analyzed by knowledge extraction engine 250 to determine if and how one or more ML models are to be updated.

At block 1030, one or more foundational ML models are updated based on the analyzed feedback. Notably, the ML models updated may include one or more ML models that were not used to create the pseudocode. The feedback may remain relevant to retraining the ML model used to create the pseudocode and one or more other ML models that are used for tangentially related purposes. At block 1040, the one or more retrained ML models are used for future processing.

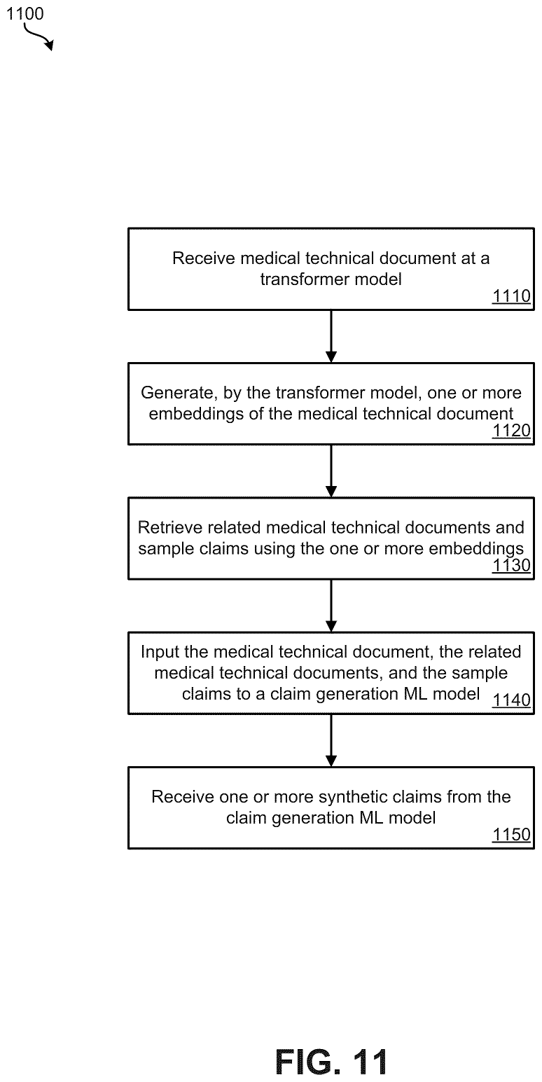

FIG. 11 illustrates an embodiment of a method 1100 of generating synthetic claims for a medical procedure based on a medical technical document. Method 1100 can be performed using the arrangements of FIGS. 1-4. Method 1100 can be performed using one or more computing systems.

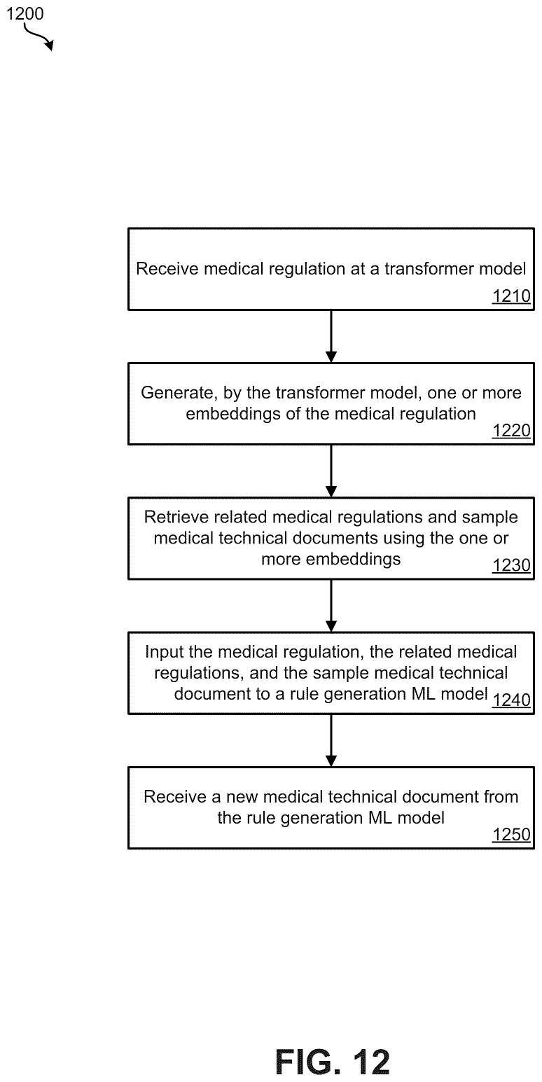

At block 1110, a medical technical document is received at a transformer model. As described above, the medical technical document may include structured and/or unstructured text that defines a context that that is required for approval of a claim that includes a request for reimbursement for a medical procedure, service, or the like. In other words, the text may define one or more criteria that are required for approving the medical procedure. For example, the one or more criteria may require that one or more medical diagnoses justifying the medical procedure be present in the claim or otherwise associated with the patient who received the medical procedure or service. As another example, the one or more criteria may require that the patient's information, such as their age, subscriber status, or the like, satisfy one or more criteria, such as a minimum or maximum age. Additionally, or alternatively, the medical technical document may define the various steps of analyzing and verifying compliance of a claim with one or more criteria defined in a medical regulation, clinical guideline, insurance policy, or the like. As further described in relation to FIG. 12, the medical technical document may be generated from one or more medical regulations, clinical guidelines, insurance policy documents, or the like.

The transformer model may be trained on healthcare contexts. For example, the transformer model may be trained using contrastive learning on pairings between similar and dissimilar medical procedures and diagnoses identified from historical medical technical documents, medical regulations, clinical guidelines, or the like. Additionally, or alternatively, the transformer model may be trained on contextually and/or semantically similar and dissimilar documents, terms, definitions, or the like. As further described in relation to FIG. 4, training the transformer model in this way can improve the ability of the transformer model to accurately generate similar embeddings for similar concepts and dissimilar embeddings for dissimilar concepts.

At block 1120, one or more embeddings of the medical technical document are generated by the transformer model. The one or more embeddings may include dense vector representations of the structured and/or unstructured text in the medical technical document. The dense vector representations may be generated from within a continuous vector space used to create embeddings of prior medical technical documents, medical regulations, or the like, as described above.

At block 1130, one or more related medical technical documents and one or more sample claims are retrieved using the one or more embeddings. The one or more related medical technical documents may include prior versions of the medical technical document received at block 1110. Additionally, or alternatively, the one or more related medical technical documents may include medical technical documents that define similar contexts required for approving the same or similar medical procedures as in the medical technical document received at block 1110.

Retrieving the one or more related medical technical documents may include performing a similarity search within an embedding datastore of medical technical documents using the one or more embeddings of the medical technical document generated at block 1120. For example, a retrieval engine, such as retrieval engine 320, may perform a nearest neighbor search, or the like, for similar embeddings. A similarity engine, such as similarity engine 322, may subsequently determine similarity scores for each of a plurality of medical technical documents that were retrieved, with the similarity score for each of the plurality being based on a comparison between the retrieved embedding and the generated embedding. The one or more related medical technical documents may then be selected based on a threshold similarity score. The embeddings of the one or more related medical technical documents may be used to retrieve the corresponding source documents, or a processed version thereof, from a separate datastore. For example, the text of the one or more related medical technical documents may be retrieved using the embeddings generated therefrom, and subsequently retrieved, as an index within a source datastore.

The one or more sample claims may be related to the one or more related medical technical documents. For example, the one or more sample claims may include synthetic and/or anonymized claims for which it is known whether they comply with the one or more related medical technical documents. The one or more sample claims may be retrieved in a similar manner as the one or more related medical technical documents. Additionally, or alternatively, the one or more sample claims may be retrieved using the embeddings of the one or more related medical technical documents.

In some embodiments, the one or more embeddings generated at block 1120 may be used to retrieve additional contextual information about the medical procedure and/or criteria defined in the medical technical document. For example, one or more procedure codes may be retrieved based on a text description of the medical procedure in the medical technical document. As another example, one or more diagnostic codes may be retrieved based on a text description in the medical technical document for diagnoses that could be used to justify the use and/or provisioning of the medical procedure and/or services.

At block 1140, the medical technical document, the one or more related medical technical documents, and the one or more sample claims are input to a claim generation ML model. As described above, each of the resources may be input to a claim generation ML model as embeddings and/or in their original formats (e.g., as structured and/or unstructured text). The claim generation ML model may be trained to generate one or more synthetic claims based on the medical technical document that can be used to test an accuracy of program code that analyzes claims for compliance with the medical technical document. For example, the one or more synthetic claims may each include a reimbursement request for the medical procedure defined in the medical technical document and/or other similar medical procedures and/or services for which the medical technical document is applicable. The one or more synthetic claims may further include some, none, or all of the required criteria defined in the medical technical document. For example, a first set of synthetic claims may be generated with the necessary details to satisfy all of the criteria while a second set of synthetic claims may be generated with one or more of the necessary details being omitted or altered so as not to satisfy the corresponding criteria.

In some embodiments, the claim generation ML model may generate multiple groups of synthetic claims when the criteria defined in the medical technical are contingent on the content of other claims. For example, consider the case of a medical technical documents that define limits on the number of times a procedure will be approved in a predefined amount of time. In this case, the claim generation ML model may generate multiple groups of synthetic claims, with each group including a synthetic claim for which an approval determination is yet to be made and one or more synthetic claims for which a determination has already been made. Groups that comply with the medical technical document may include fewer numbers of claims for the same procedure than the limit defined in the medical technical document with or without additional claims for the medical procedure that are sufficiently old so as not to count against the limit.

At block 1150, one or more synthetic claims are received from the claim generation ML model. As described above, the one or more synthetic claims may be usable to test an accuracy of program code designed to analyze claims for compliance with the medical technical document. For example, and as described above, the one or more synthetic claims may include a first group of synthetic claims that satisfy the required context as defined in the medical technical document and a second group of synthetic claims that do not satisfy the required context. As further described herein, the synthetic claims may be generated with labels indicating whether or not they satisfy the context. Such labels may further indicate the reason why they do or do not satisfy the context. For example, the labels may specify the criteria in the medical technical document that are not satisfied within the synthetic claims that do not satisfy the context.

In some embodiments, the method further includes executing the program code on the one or more synthetic claims to verify the accuracy of the program code. For example, the results of the analysis performed by the program code may be compared with the labels generated with the synthetic claims to produce a score representing the accuracy of the program code.

FIG. 12 illustrates an embodiment of a method 1200 of generating a medical technical document based on a medical regulation. Method 1200 can be performed using the arrangements of FIGS. 1-4. Method 1200 can be performed using one or more computing systems.

At block 1210, a medical regulation is received at a transformer model. As further described above, the medical regulation may include structure and/or unstructured text that outlines various rules, conditions, and criteria for approving payments for medical procedures, services, or the like. The medical regulation may be received as a text document and preprocessed as described above in relation to FIG. 5 before being provided to the transformer model. The transformer model may be the same, or function in a similar manner, as described above in relation to method 1100.

At block 1220, one or more embeddings of the medical regulation are generated by the transformer model. As further described above, each of the one or more embeddings of the medical regulation may represent uniquely identifiable features of the medical regulation. For example, the one or more embeddings may include an embedding for the title of the document, one or more embeddings for the structural components of the document (e.g., headings and subheadings), one or more embeddings for discrete portions of text, or the like.

At block 1230, related medical regulations and sample medical technical documents are retrieved using the one or more embeddings. The related medical regulations and sample medical technical documents may be retrieved in a similar manner as described above in relation to block 1130 of method 1100. For example, one or more similarity searches within an embedding datastore of medical regulations may be performed to retrieve one or more related medical regulations.

At block 1240, the medical regulation, the related medical regulations, and the sample medical technical documents are input to a rule generation ML model. The rule generation ML model may be trained to generate medical technical documents from medical regulations. For example, given a medical regulation, the rule generation ML model may generate one or more medical technical documents, each corresponding to a different set of rules or conditions defined in the medical regulation. The rule generation ML model may use the related medical regulations and corresponding sample medical technical documents as examples from which to generate the new medical technical document.

At block 1250, a new medical technical document is received from the rule generation ML model. As described above, the new medical technical document may correspond to a portion of the medical regulation and include structure text that defines the one or more criteria required to approve claims for medical procedures covered in the portion of the medical regulation. In some embodiments, the new medical technical document is subsequently used to generate synthetic claims, as described above in relation to method 1100. Additionally, or alternatively, the new medical technical document may be used to generate program code that analyzes claims for compliance with the medical regulation and/or medical technical document.

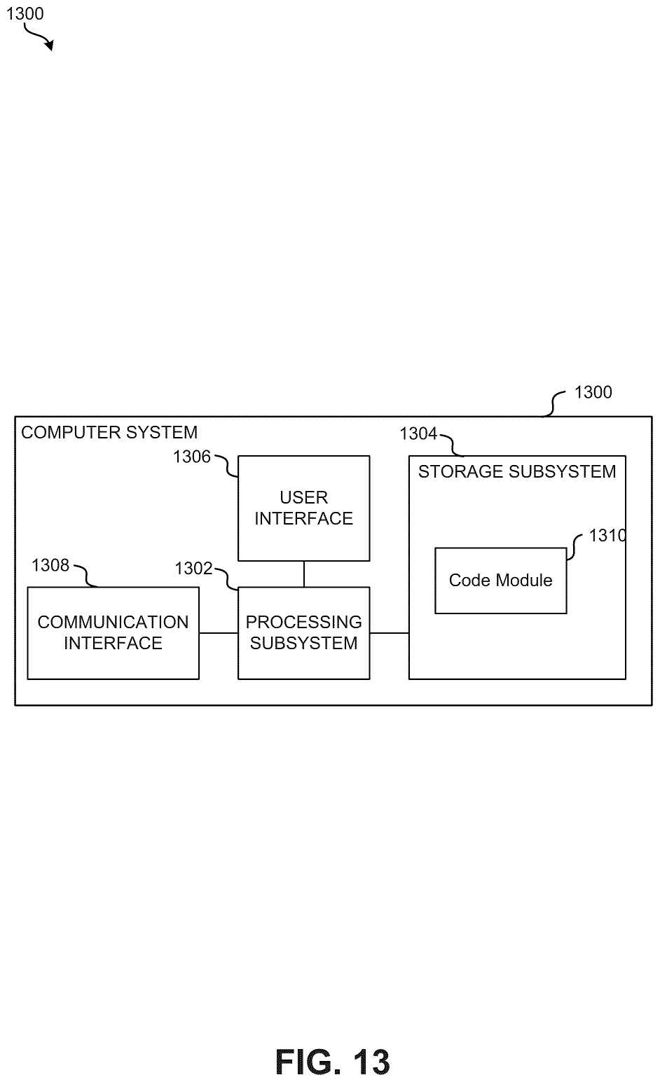

In some embodiments, one or more systems for generating pseudocode and test data, such as system 200 and system 300, are implemented on a computer system. FIG. 13 illustrates a block diagram of an embodiment of a computer system 1300. Computer system 1300 can implement some or all functions, behaviors, and/or capabilities described above that would use electronic storage or processing, as well as other functions, behaviors, or capabilities not expressly described. Computer system 1300 includes a processing subsystem 1302, a storage subsystem 1304, a user interface 1306, and/or a communication interface 1308. Computer system 1300 can also include other components (not explicitly shown) such as a battery, power controllers, and other components operable to provide various enhanced capabilities. In various embodiments, computer system 1300 can be implemented in a desktop or laptop computer, mobile device (e.g., tablet computer, smart phone, mobile phone), wearable device, media device, application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, or electronic units designed to perform a function or combination of functions described above.

Storage subsystem 1304 can be implemented using a local storage and/or removable storage medium, e.g., using disk, flash memory (e.g., secure digital card, universal serial bus flash drive), or any other non-transitory storage medium, or a combination of media, and can include volatile and/or nonvolatile storage media. Local storage can include random access memory (RAM), including dynamic RAM (DRAM), static RAM (SRAM), or battery backed up RAM. In some embodiments, storage subsystem 1304 can store one or more applications and/or operating system programs to be executed by processing subsystem 1302, including programs to implement some or all operations described above that would be performed using a computer. For example, storage subsystem 1304 can store one or more code modules 1310 for implementing one or more method steps described above.

A firmware and/or software implementation may be implemented with modules (e.g., procedures, functions, and so on). A machine-readable medium tangibly embodying instructions may be used in implementing methodologies described herein. Code modules 1310 (e.g., instructions stored in memory) may be implemented within a processor or external to the processor. As used herein, the term “memory” refers to a type of long term, short term, volatile, nonvolatile, or other storage medium and is not to be limited to any particular type of memory or number of memories or type of media upon which memory is stored.

Moreover, the term “storage medium” or “storage device” may represent one or more memories for storing data, including read only memory (ROM), RAM, magnetic RAM, core memory, magnetic disk storage mediums, optical storage mediums, flash memory devices and/or other machine-readable mediums for storing information. The term “machine-readable medium” includes, but is not limited to, portable or fixed storage devices, optical storage devices, wireless channels, and/or various other storage mediums capable of storing instruction(s) and/or data.

Furthermore, embodiments may be implemented by hardware, software, scripting languages, firmware, middleware, microcode, hardware description languages, and/or any combination thereof. When implemented in software, firmware, middleware, scripting language, and/or microcode, program code or code segments to perform tasks may be stored in a machine-readable medium such as a storage medium. A code segment (e.g., code module 1310) or machine-executable instruction may represent a procedure, a function, a subprogram, a program, a routine, a subroutine, a module, a software package, a script, a class, or a combination of instructions, data structures, and/or program statements. A code segment may be coupled to another code segment or a hardware circuit by passing and/or receiving information, data, arguments, parameters, and/or memory contents. Information, arguments, parameters, data, etc., may be passed, forwarded, or transmitted by suitable means including memory sharing, message passing, token passing, network transmission, etc.

Implementations of the techniques, blocks, steps and means described above may be done in various ways. For example, these techniques, blocks, steps and means may be implemented in hardware, software, or a combination thereof. For a hardware implementation, the processing units may be implemented within one or more ASICs, DSPs, DSPDs, PLDs, FPGAs, processors, controllers, micro-controllers, microprocessors, other electronic units designed to perform the functions described above, and/or a combination thereof.

Each code module 1310 may comprise sets of instructions (codes) embodied on a computer-readable medium that directs a processor of a computer system 1300 to perform corresponding actions. The instructions may be configured to run in sequential order, in parallel (such as under different processing threads), or in a combination thereof. After loading a code module 1310 on a general purpose computer system, the general purpose computer is transformed into a special purpose computer system.

Computer programs incorporating various features described herein (e.g., in one or more code modules 1310) may be encoded and stored on various computer readable storage media. Computer readable media encoded with the program code may be packaged with a compatible electronic device, or the program code may be provided separately from electronic devices (e.g., via Internet download or as a separately packaged computer readable storage medium). Storage subsystem 1304 can also store information useful for establishing network connections using the communication interface 1308.

User interface 1306 can include input devices (e.g., touch pad, touch screen, scroll wheel, click wheel, dial, button, switch, keypad, microphone, etc.), as well as output devices (e.g., video screen, indicator lights, speakers, headphone jacks, virtual- or augmented-reality display, etc.), together with supporting electronics (e.g., digital to analog or analog to digital converters, signal processors, etc.). A user can operate input devices of user interface 1306 to invoke the functionality of computer system 1300 and can view and/or hear output from computer system 1300 via output devices of user interface 1306. For some embodiments, the user interface 1306 might not be present (e.g., for a process using an ASIC).

Processing subsystem 1302 can be implemented as one or more processors (e.g., integrated circuits, one or more single core or multi core microprocessors, microcontrollers, central processing unit, graphics processing unit, etc.). In operation, processing subsystem 1302 can control the operation of computer system 1300. In some embodiments, processing subsystem 1302 can execute a variety of programs in response to program code and can maintain multiple concurrently executing programs or processes. At a given time, some or all of a program code to be executed can reside in processing subsystem 1302 and/or in storage media, such as storage subsystem 1304. Through programming, processing subsystem 1302 can provide various functionality for computer system 1300. Processing subsystem 1302 can also execute other programs to control other functions of computer system 1300, including programs that may be stored in storage subsystem 1304.

Communication interface 1308 can provide voice and/or data communication capability for computer system 1300. In some embodiments, communication interface 1308 can include radio frequency (RF) transceiver components for accessing wireless data networks (e.g., Wi-Fi network; 3G, 4G/LTE; etc.), mobile communication technologies, components for short range wireless communication (e.g., using Bluetooth communication standards, near-field communications (NFC), etc.), other components, or combinations of technologies. In some embodiments, communication interface 1308 can provide wired connectivity (e.g., universal serial bus, Ethernet, universal asynchronous receiver/transmitter, etc.) in addition to, or in lieu of, a wireless interface. Communication interface 1308 can be implemented using a combination of hardware (e.g., driver circuits, antennas, modulators/demodulators, encoders/decoders, and other analog and/or digital signal processing circuits) and software components. In some embodiments, communication interface 1308 can support multiple communication channels concurrently. In some embodiments, the communication interface 1308 is not used.

It will be appreciated that computer system 1300 is illustrative and that variations and modifications are possible. A computing device can have various functionality not specifically described (e.g., voice communication via cellular telephone networks) and can include components appropriate to such functionality.

Further, while the computer system 1300 is described with reference to particular blocks, it is to be understood that these blocks are defined for convenience of description and are not intended to imply a particular physical arrangement of component parts. For example, the processing subsystem 1302, the storage subsystem 1304, the user interface 1306, and/or the communication interface 1308 can be in one device or distributed among multiple devices.

Further, the blocks need not correspond to physically distinct components. Blocks can be configured to perform various operations, e.g., by programming a processor or providing appropriate control circuitry, and various blocks might or might not be reconfigurable depending on how an initial configuration is obtained. Embodiments of the present invention can be realized in a variety of apparatus including electronic devices implemented using a combination of circuitry and software. Electronic devices described herein can be implemented using computer system 1300.

It should be noted that the methods, systems, and devices discussed above are intended merely to be examples. It must be stressed that various embodiments may omit, substitute, or add various procedures or components as appropriate. For instance, it should be appreciated that, in alternative embodiments, the methods may be performed in an order different from that described, and that various steps may be added, omitted, or combined. Also, features described with respect to certain embodiments may be combined in various other embodiments. Different aspects and elements of the embodiments may be combined in a similar manner. Also, it should be emphasized that technology evolves and, thus, many of the elements are examples and should not be interpreted to limit the scope of the invention.

Specific details are given in the description to provide a thorough understanding of the embodiments. However, it will be understood by one of ordinary skill in the art that the embodiments may be practiced without these specific details. For example, well-known, processes, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing embodiments of the invention. Various changes may be made in the function and arrangement of elements without departing from the spirit and scope of the invention.

Also, it is noted that the embodiments may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure.

Having described several embodiments, it will be recognized by those of skill in the art that various modifications, alternative constructions, and equivalents may be used without departing from the spirit of the invention. For example, the above elements may merely be a component of a larger system, wherein other rules may take precedence over or otherwise modify the application of the invention. Also, a number of steps may be undertaken before, during, or after the above elements are considered. Accordingly, the above description should not be taken as limiting the scope of the invention.

What is claimed is:

1. A method of generating synthetic claims for medical procedures, comprising:
receiving a medical technical document comprising text data that defines a context that is required for approval of a medical procedure at a transformer model executed on a computer system, wherein the transformer model is trained on healthcare contexts;
generating, by the transformer model executed on the computer system, one or more embeddings of the medical technical document;
retrieving, by the computer system, using the one or more embeddings, one or more related medical technical documents and one or more sample claims, wherein the one or more sample claims are related to the related medical technical documents;
inputting, by the computer system, to a claim generation machine learning model, the medical technical document, the one or more related medical technical documents, and the one or more sample claims to generate one or more synthetic claims that each comprise a reimbursement request for the medical procedure; and
receiving, by the computer system from the claim generation machine learning model, the one or more synthetic claims, wherein the one or more synthetic claims are usable to test an accuracy of program code that analyzes claims for compliance with the medical technical document.

2. The method of generating synthetic claims for medical procedures of claim 1, wherein the one or more embeddings generated by the transformer model are dense vector representations in a continuous vector space.

3. The method of generating synthetic claims for medical procedures of claim 1, wherein retrieving the one or more related medical technical documents comprises performing a similarity search within an embedding datastore of medical technical documents using the one or more embeddings of the medical technical document.

4. The method of generating synthetic claims for medical procedures of claim 1, wherein retrieving the one or more related medical technical documents comprises:
determining a similarity score for each of a plurality of medical technical documents, wherein the similarity score for a respective medical technical document of the plurality of medical technical documents is based on a comparison between the one or more embeddings of the medical technical document and one or more embeddings of the respective medical technical document; and
selecting the one or more related medical technical documents from the plurality of medical technical documents based on a threshold similarity score.

5. The method of generating synthetic claims for medical procedures of claim 1, wherein the transformer model is trained using contrastive learning on pairings between similar and dissimilar medical procedures and diagnoses identified from historical medical technical documents.

6. The method of generating synthetic claims for medical procedures of claim 1, wherein the context defines one or more criteria that are required for approval of the medical procedure.

7. The method of generating synthetic claims for medical procedures of claim 6, wherein the one or more criteria comprise a first medical diagnosis that justifies the medical procedure.

8. The method of generating synthetic claims for medical procedures of claim 7, wherein the one or more synthetic claims comprise a first claim that justifies the medical procedure with the first medical diagnosis.

9. The method of generating synthetic claims for medical procedures of claim 8, further comprising:
retrieving, using the one or more embeddings, a procedure code for the medical procedure and a diagnostic code for the medical diagnosis; and
providing the procedure code and the diagnostic code to the claim generation machine learning model.

10. The method of generating synthetic claims for medical procedures of claim 7, wherein the one or more synthetic claims comprise a first claim that justifies the medical procedure with a second medical diagnosis that is not included in the one or more criteria.

11. The method of generating synthetic claims for medical procedures of claim 1, wherein:
the one or more synthetic claims comprise a first set of synthetic claims that satisfy the context and a second set of synthetic claims that do not satisfy the context; and
each of the one or more synthetic claims is generated with a label indicating whether it satisfies the context or not.

12. The method of generating synthetic claims for medical procedures of claim 11, further comprising:
executing the program code on the one or more synthetic claims to produce determinations, for each respective synthetic claim of the one or more synthetic claims, indicative of whether the respective synthetic claim complies with the medical technical document; and
determining, based on a comparison between the results generated by the program code and the label generated with each of the one or more synthetic claims, the accuracy of the program code.

13. A retrieval augmented synthetic test claim generation system, comprising:
one or more processors; and
a computer-readable storage media storing computer-executable instructions that, when executed by the one or more processors, cause the retrieval augmented synthetic test claim generation system to:
receive a medical technical document comprising text data that defines a context that is required for approval of a medical procedure at a transformer model, wherein the transformer model is trained on healthcare contexts;
generate, by the transformer model, one or more embeddings of the medical technical document;
retrieve, using the one or more embeddings, one or more related medical technical documents and one or more sample claims, wherein the one or more sample claims are related to the related medical technical documents;
input, to a claim generation machine learning model, the medical technical document, the one or more related medical technical documents, and the one or more sample claims to generate one or more synthetic claims that each comprise a reimbursement request for the medical procedure; and
receive, from the claim generation machine learning model, the one or more synthetic claims, wherein the one or more synthetic claims are usable to test an accuracy of program code that analyzes claims for compliance with the medical technical document.

14. The retrieval augmented synthetic test claim generation system of claim 13, wherein the one or more embeddings generated by the transformer model are dense vector representations in a continuous vector space.

15. The retrieval augmented synthetic test claim generation system of claim 13, wherein retrieving the one or more related medical technical documents comprises:
determining a similarity score for each of a plurality of medical technical documents, wherein the similarity score for a respective medical technical document of the plurality of medical technical documents is based on a comparison between the one or more embeddings of the medical technical document and one or more embeddings of the respective medical technical document; and
selecting the one or more related medical technical documents from the plurality of medical technical documents based on a threshold similarity score.

16. The retrieval augmented synthetic test claim generation system of claim 13, wherein:
the one or more synthetic claims comprise a first set of synthetic claims that satisfy the context and a second set of synthetic claims that do not satisfy the context; and
each of the one or more synthetic claims is generated with a label indicating whether it satisfies the context or not.

17. One or more non-transitory computer-readable storage media storing one or more instructions which, when executed by one or more processors of a retrieval augmented synthetic test claim generation system, cause the one or more processors to:
receive a medical technical document comprising text data that defines a context that is required for approval of a medical procedure at a transformer model, wherein the transformer model is trained on healthcare contexts;
generate, by the transformer model, one or more embeddings of the medical technical document;
retrieve, using the one or more embeddings, one or more related medical technical documents and one or more sample claims, wherein the one or more sample claims are related to the related medical technical documents;
input, to a claim generation machine learning model, the medical technical document, the one or more related medical technical documents, and the one or more sample claims to generate one or more synthetic claims that each comprise a reimbursement request for the medical procedure; and
receive, from the claim generation machine learning model, the one or more synthetic claims, wherein the one or more synthetic claims are usable to test an accuracy of program code that analyzes claims for compliance with the medical technical document.

18. The one or more non-transitory computer-readable storage media of claim 17, wherein the one or more embeddings generated by the transformer model are dense vector representations in a continuous vector space.

19. The one or more non-transitory computer-readable storage media of claim 17, wherein retrieving the one or more related medical technical documents comprises performing a similarity search within an embedding datastore of medical technical documents using the one or more embeddings of the medical technical document.

20. The one or more non-transitory computer-readable storage media of claim 17, wherein the transformer model is trained using contrastive learning on pairings between similar and dissimilar medical procedures and diagnoses identified from historical medical technical documents.
